(19) United States
(12) Patent Application Publication
(10) Pub. No.: US 2010/0240770 A1
(43) Pub. Date: Sep. 23, 2010

(54) Synthesis and use of colloidal III-V nanoparticles

(76) Inventors: Jifa Qi, West Roxbury, MA (US); Angela M. Belcher, Lexington, MA (US); Amy Shi, Cambridge, MA (US); Saeeda Jaffar, New York, NY (US)

Correspondence Address: STEPTOE & JOHNSON LLP, 1330 CONNECTICUT AVENUE, N.W., WASHINGTON, DC, 20036, US

(21) Appl. No.: 11/375,586

(22) Filed: Mar. 13, 2006

Related U.S. Application Data
(60) Provisional application No. 60/660,568, filed on Mar. 11, 2005.

(51) Int. Cl. A61K 47/02; A61P 35/00; A61P 31/18; C01B 21/06; C01B 21/072; C01B 21/00; B32B 1/00; H01B 1/02

(52) U.S. Cl. 514/769; 423/409; 423/412; 423/351; 428/403; 428/402; 252/518.1; 977/773

(57) ABSTRACT

A colloidal suspension of III-V semiconductor nanoparticles.

This application claims priority to U.S. Provisional Application No. 60/660,568, filed Mar. 11, 2005, the entire contents of which are incorporated herein by reference.

This invention was made under a DARPA Joint ATO/DSO Grant. The United States Government may have certain rights in this invention.

### FIELD OF THE INVENTION

This invention relates to a method of synthesis of III-V nanoparticles and uses of the nanoparticles.

### BACKGROUND OF THE INVENTION

In recent years, group III-nitride materials such as GaN have rapidly gained prominence as efficient optical materials for light emitting and laser diodes that can span the UV to visible wavelength regimes (F. A. Ponce, et al., Nature (1997) 386, 351; S. Nakamura, et al. The Blue Laser Diode, Springer, Berlin 1997; J. W. Orton, et al., Rep. Prog. Phys. (1998) 61, 1; S. Nakamura, J. Mater. Res. (1999) 14, 2716; S. C. Jain, et al., J. Appl. Phys. (2000) 87, 965), as well as for potential use in solar cells (J. Wu, et al., Superlattices and Microstructures, 2003, 34: 63-75). GaN heterostructure devices have been formed through epitaxial growth techniques such as metal-organic chemical vapor deposition (MOCVD) or Molecular Beam Epitaxy (MBE) (S. Nakamura, et al., Jpn. J. Appl. Phys. Part 2 (1995) 34, L797). The formation of optically efficient colloidal nanoparticles of GaN has not been extensively explored. A solution-based synthesis would provide a path for nanoparticle-based self-assembly and layer-by-layer deposition of GaN nanoparticles with non-lattice matched materials. The freedom to grow GaN nanoparticles free from lattice matched surfaces and catalysts and combine them with other materials would enable new optical devices and efficient phosphors and could also extend the range of the III-nitride and III-phosphide materials to additional applications such as in vivo imaging of biological systems.

### DEFINITIONS

“Alkyl”: The term “alkyl” as used herein refers to saturated, straight- or branched-chain hydrocarbon radicals derived from a hydrocarbon moiety containing between one and twenty carbon atoms by removal of a single hydrogen atom. Examples of alkyl radicals include, but are not limited to, methyl, ethyl, propyl, isopropyl, n-butyl, tert-butyl, n-pentyl, neopentyl, n-hexyl, n-heptyl, n-octyl, n-decyl, n-undecyl, and dodecyl.

“Biologically active agents”: As used herein, “biologically active agents” is used to refer to compounds or entities that alter, inhibit, activate, or otherwise affect biological or chemical events. For example, biomolecules may be biologically active agents. In another example, biologically active agents may include, but are not limited to, anti-AIDS substances, anti-cancer substances, antibiotics, immunosuppressants, anti-viral substances, enzyme inhibitors, neurotoxins, opioids, hypnotics, anti-histamines, lubricants, tranquilizers, anti-convulsants, muscle relaxants and anti-Parkinson substances, anti-spasmodics and muscle contractants including channel blockers, miotics and anti-cholinergics, anti-glaucoma compounds, anti-parasite and/or anti-protozoal compounds, modulators of cell-extracellular matrix interactions including cell growth inhibitors and anti-adhesion molecules, vasodilating agents, inhibitors of DNA, RNA or protein synthesis, anti-hypertensives, analgesics, anti-pyretics, steroidal and non-steroidal anti-inflammatory agents, anti-angiogenic factors, anti-secretory factors, anticoagulants and/or antithrombotic agents, local anesthetics, ophthalmics, prostaglandins, anti-depressants, anti-psychotic substances, anti-emetics, and imaging agents. In certain embodiments, the bioactive agent is a drug. Preferably, though not necessarily, the drug is one that has already been deemed safe and effective for use by the appropriate governmental agency or body. For example, drugs for human use listed by the FDA under 21 C.F.R. §§330.5, 331 through 361, and 440 through 460; drugs for veterinary use listed by the FDA under 21 C.F.R. §§500 through 589, incorporated herein by reference, are all considered acceptable for use in accordance with the present invention.

A more complete listing of bioactive agents and specific drugs suitable for use in the present invention may be found in “Pharmaceutical Substances: Syntheses, Patents, Applications” by Axel Kleemann and Jurgen Engel, Thieme Medical Publishing, 1999; the “Merck Index: An Encyclopedia of Chemicals, Drugs, and Biologicals”, Edited by Susan Budavari et al., CRC Press, 1996, and the United States Pharmacopeia-25/National Formulary-20, published by the United States Pharmcopeial Convention, Inc., Rockville Md., 2001, all of which are incorporated herein by reference.

“Biomolecules”: The term “biomolecules”, as used herein, refers to molecules (e.g., proteins, amino acids, peptides, polynucleotides, nucleotides, carbohydrates, sugars, lipids, nucleoproteins, glycoproteins, lipoproteins, steroids, etc.) whether naturally-occurring or artificially created (e.g., by synthetic or recombinant methods) that are commonly found in cells and tissues. Specific classes of biomolecules include, but are not limited to, enzymes, receptors, neurotransmitters, hormones, cytokines, cell response modifiers such as growth factors and chemotactic factors, antibodies, vaccines, haptens, toxins, interferons, ribozymes, anti-sense agents, plasmids, DNA, and RNA.

“Polynucleotide”, “nucleic acid”, or “oligonucleotide”: The terms “polynucleotide”, “nucleic acid”, or “oligonucleotide” refer to a polymer of nucleotides. The terms “polynucleotide”, “nucleic acid”, and “oligonucleotide”, may be used interchangeably. Typically, a polynucleotide comprises at least two nucleotides. DNAs and RNAs are polynucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, 2′-methoxyribose, 2′-aminoribose, ribose, 2′-deoxyribose, arabinose, and hexose), or modified phosphate groups (e.g., phosphorothioates and 5′-N phosphoramidite linkages). Enantiomers of natural or modified nucleosides may also be used. Nucleic acids also include nucleic acid-based therapeutic agents, for example, nucleic acid ligands, siRNA, short hairpin RNA, antisense oligonucleotides, ribozymes, aptamers, and SPIEGELMERS™, oligonucleotide ligands described in Wlotzka, et al., Proc. Nat'l. Acad. Sci. USA, 2002, 99(13):8898, the entire contents of which are incorporated herein by reference.

“Polypeptide”, “peptide”, or “protein”: According to the present invention, a “polypeptide”, “peptide”, or “protein” comprises a string of at least three amino acids linked together by peptide bonds. The terms “polypeptide”, “peptide”, and “protein”, may be used interchangeably. Peptide may refer to an individual peptide or a collection of peptides. Inventive peptides preferably contain only natural amino acids, although non natural amino acids (i.e., compounds that do not occur in nature but that can be incorporated into a polypeptide chain) and/or amino acid analogs as are known in the art may alternatively be employed. Also, one or more of the amino acids in a peptide may be modified, for example, by the addition of a chemical entity such as a carbohydrate group, a phosphate group, a farnesyl group, an isofarnesyl group, a fatty acid group, a linker for conjugation, functionalization, or other modification, etc. In one embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide.

“Polysaccharide”, “carbohydrate” or “oligosaccharide”: The terms “polysaccharide”, “carbohydrate”, or “oligosaccharide” refer to a polymer of sugars. The terms “polysaccharide”, “carbohydrate”, and “oligosaccharide”, may be used interchangeably. Typically, a polysaccharide comprises at least two sugars. The polymer may include natural sugars (e.g., glucose, fructose, galactose, mannose, arabinose, ribose, and xylose) and/or modified sugars (e.g., 2′-fluororibose, 2′-deoxyribose, and hexose).

### SUMMARY OF THE INVENTION

In one aspect, the invention is a method of producing colloidal III-V semiconductor crystals. The method includes reacting a solution comprising at least one source material including a Group III element, a source material including a Group V element, and a reducing agent for a predetermined time period at a predetermined temperature, wherein the source material is a salt of a Group III element, a mono, di, or trialkyl compound of a group V element, or a chelate of a Group III element with a mono-, di-, or trialkyl compound of a group V element. The at least one Group III element may be gallium, indium, aluminum, or boron. The Group V element may be nitrogen, phosphorus, arsenic, or antimony. The predetermined temperature may be about 100° C. to about 450° C., and the predetermined time period may be between 8 and 16 hours. The solution may further include a source material of a rare earth element or a transition metal, wherein the a salt of the rare earth or transition metal element or a chelate of the rare earth or transition metal element with a mono-, di-, or trialkyl compound of the group V element. The solution may include source materials for a first Group III element and a second Group III element. The source material for the Group III element and the source material for the Group V element may be the same material.

The solution may include a solvent, and the source material for the Group V element may be the solvent. The solvent may be triethylamine, acetonitrile, chloroform, benzene, paraffin oil, or naphthalene. The solution may further include a capping agent, for example, TOPO, polyallylamine, hyaluronic acid, acetamidine hydrochloride, cetyltrimethyl ammonium bromide, benzalkonium chloride, poly(vinylsulfonic acid), linear and branched poly(ethylene imine) PEI, polyallylamine HCl (PAH), polylysine, chitosan, poly(diallydimethylammonium chloride) (PDAC), a polysaccharide, a polymer of positively charged amino acids, polyaminoserinate, hyaluronan, polymalic acid, a polyimide, phenylalanine, histidine, hexahistidine, serine, proline, a polymer of negatively charged or acidic amino acids, a phospholipid, a PEG-derivitized phospholipid, a polynucleotide, or an amino acid oligomer. The capping agent may be A-R-X, wherein A is thiol, phosphine, phosphine oxide, amine, amide oxide, sulfonate, carbonate, or carboxylate, R is straight or branched alkane optionally comprising amide, ketone, ether, or aryl, and X is hydroxyl, amine, amide, carboxylate, sulfonate, phosphate, or ammonium.

The method may further include condensing the solution by removing at least a portion of the solvent and heating the remaining product at a temperature between about 300° C. and 450° C. for about 8 to about 16 hours. The method may further include recovering III-V nanoparticles from the solution, suspending the nanoparticles in a solvent with a source material including a predetermined Group III element and a source material including a predetermined Group V element, and holding the suspension at a predetermined temperature for a predetermined period of time, wherein a layer of a semiconductor material including the Group III element and the Group V element forms on the nanoparticle. The method may further include covalently or non-covalently attaching a biologically active agent or a targeting agent to the nanoparticles.

In another aspect, the invention is a method of patterning nanoparticles on a surface. The method includes producing colloidal III-V semiconductor crystals, capping the III-V semiconductor crystals with a material having a predetermined charge, providing a substrate having a charged material patterned thereon, the charged material having a charge opposite that of the predetermined charge, and incubating the substrate with the capped III-V semiconductor crystals. Providing a substrate may include patterning the charged material on the substrate. The charged material may be a SAM-forming material or one of TOPO, polyallylalanine, hyaluronic acid, acetamidine hydrochloride, cetyltrimethyl ammonium bromide, benzalkonium chloride, poly(vinylsulfonic acid), linear and branched poly(ethylene imine) PEI, polyallylamine HCl (PAH), polylysine, chitosan, poly(diallydimethylammonium chloride) (PDAC), polysaccharides, polymers of positively charged amino acids, polyaminoserinate, hyaluronan, polymalic acid, polyimides, polymers of negatively charged or acidic amino acids, and polynucleotides.

In another aspect, the invention is a core shell structure including a core of a first III-V semiconductor material including a first Group III element and a first Group V element and a layer of a second III-V semiconductor material including a second Group III element and a second Group V element. The first and second Group III elements may be the same or the first and second Group V elements may be the same. The first and second Group III elements may be different and the first and second Group V elements may be different. The core shell structure may further include a capping layer disposed on the surface of the core shell structure. The core may be substantially GaN and the shell may be substantially InN, or vice versa. Each of the core and shell may be independently selected from AlP, AlAs, AlSb, AlN, GaN, GaP, GaAs, GaSb, InP, InAs, InSb, and InN. The thickness of the shell may be about the same as the radius of the core. For example, the ratio of the shell thickness and the core radius may be between about 1:1 and 1:5, about 1:1 and 1:4, about 1:1 and 1:3, or about 1:1 and 1:2.

In another aspect, the invention is a III-V semiconductor nanoparticle including a first Group III element and a second Group III element in a predetermined ratio and a Group V element. The nanoparticle diameter may be between about 2 and about 15 nm, e.g., between about 5 and about 7 nm.

In another aspect, the invention is a population of a plurality of III-V semiconductor nanoparticles, wherein the variation of the particle diameter is about 15% or less, for example, about 10% or less, or about 5% or less. In another aspect, the invention is a colloidal solution of III-V semiconductor nanoparticles.

In another aspect, the invention is a colloidal solution of Group III-nitride semiconductor crystals. The Group III element may be one or two of Ga, Al, In, and B. The semiconductor crystals may be between about 2 and about 15 nm in diameter or between about 15 and about 30 nm in diameter. The semiconductor crystals may include a capping agent. The semiconductor crystals may be conjugated to a biologically active agent. The semiconductor crystals may be water soluble.

In another aspect, the invention is a colloidal solution of substantially spherical III-V semiconductor crystals, wherein the variation in particle diameter of the crystals is about 15% or less, for example, about 10% or less, or about 5% or less.

### BRIEF DESCRIPTION OF THE DRAWING

The invention is described with reference to the several figures of the drawing, in which,

FIG. 1 is a (A) transmission electron micrograph and (B) XRD pattern of Ga0.95In0.5N nanoparticles produced according to an exemplary embodiment of the invention.

FIG. 2 is a series of XPS spectra of Ga0.95In0.5N nanoparticles produced according to an exemplary embodiment of the invention.

FIG. 3 is a transmission electron micrograph of (InN)GaN core shell nanoparticles produced according to an exemplary embodiment of the invention.

FIG. 4A is a graph showing the surface charge on bare GaN (UC) nanoparticles and nanoparticles capped with histidine (His), phenylalanine (Phe), proline (Pro), and serine (Ser).

FIG. 4B is a graph showing the surface charge of GaN nanoparticles coated with poly(allyl amine) and bovine serum albumin on PAA.

FIG. 4C is a photograph of GaN nanoparticles coated with, from left to right, TOPO, His, Phe, Pro, and Ser under illumination by a UV lamp at 365 nm.

FIG. 4D is a microfluorescence image of cationic PAA-coated GaN nanoparticles deposited on a HA patterned substrate taken through a TRITC filter. The blue areas correspond to the nanoparticle layers and the dark areas are the GaN non-binding regions (scale bar 50 micrometers).

FIG. 5 is a schematic of a water soluble GaN nanoparticle according to an exemplary embodiment of the invention.

FIG. 6 is a set of transmission electron micrographs of InN nanoparticles produced according to an exemplary embodiment of the invention; the crystals are about 2-3 nm in diameter.

FIG. 7 is a set of XRD spectra of (A) GaN and (B) InN nanoparticles produced according to an exemplary embodiment of the invention.

FIG. 8 is a (A) XRD pattern and (B) transmission electron micrograph of AlN nanoparticles produced according to an exemplary embodiment of the invention.

FIG. 9A is a bright field TEM image of GaN nanoparticles produced at 350.

FIG. 9B is a high resolution TEM image of an individual GaN nanoparticle.

FIG. 9C is a selected area electron diffraction pattern of the GaN nanoparticle of FIG. 9B. The rings numbered 1-5 correspond to the (100), (101), (102), (110), and (200) planes of wurtzite structured GaN.

FIG. 9D is a series of x-ray diffraction patterns of GaN particles produced at (a) 200° C., (b) 350° C., and (c) 450° C., respectively. The expected peak positions of (d) zincblende and (d) wurtzite structured GaN (JCPDS #520791 and 500792, respectively) are also shown.

FIG. 9E is a graph showing the optical absorption and photoluminescence spectrum of GaN nanoparticles.

FIG. 10 is a series of TEM images of GaN nanoparticles; produced at (A) 200° C., (B) 350° C., and (C) 450° C.

FIG. 11 is a series of XPS spectra of InN, GaN, and (InN)GaN core shell nanoparticles produced according to various exemplary embodiments of the invention.

FIG. 12A is a series of graphs showing the atomic ratios of Ga to In as (A) (GaN)InN and (B-D) (InN)GaN core shell structures are etched.

FIG. 12B is a series of photographs of a GaN nanoparticle/phage hybrid film viewed under room and UV illumination.

FIG. 13 is a set of polarized optical microscope images of hybrid GaN/phage films.

FIG. 14 is a series of atomic force microscope images of GaN nanoparticle/pVIII phage hybrid films.

FIG. 15 is a series of atomic force microscope images of pVIII type M13 phage templated GaN nanowires.

FIG. 16 is a series of electron transmission electron micrographs of pVIII type M13 phage templated GaInN nanowires.

FIG. 17 is a series of TEM images of pVIII type M13 phage templated GaN and InN nanowires.

FIG. 18 is a series of micrographs of COS-7 monkey kidney epithelium cells after incubation with GaN nanoparticles using A) phase contrast, B) a DAPI filter to show the GaN nanoparticles, C) a TRITC filter. FIG. 7D is a merged image of FIGS. 7B and C to co-localize GaN nanoparticles and endosomes (scale bar=50 micrometers).

### DETAILED DESCRIPTION OF CERTAIN PREFERRED EMBODIMENTS

In one embodiment, optically active colloidal III-V nanoparticles are synthesized in solution. The nanoparticles may be capped using bio-mediated methods and incorporated into cells, formed into planar-patterned structures, or formed into films. The freestanding nature of the individual nanoparticles coupled with the ability to functionalize them with a wide variety of capping ligands provides a platform for interaction of III-V nanoparticles with cells, many material surfaces and facilitates a new approach to self-assemble III-V nanoparticles.

KBH₄ and its derivatives have been used to prepare finely divided powders of metals and alloys by reducing metal salts in organic solvents (H. Bönnemann, et al., Angew. Chem. Int. Ed. Engl. (1990) 29, 273) and also for InP nanoparticles (P. Yan, et al., J. Mater. Chem. (1999) 9, 1831). We have extended this method to prepare metal nitride compounds. TOPO has been commonly used as a capping agent for the syntheses of CdSe and InP nanoparticles (C. B. Murray, et al., J. Am. Chem. Soc. (1993) 115, 8706; A. A. Guzelian, et al., J. Phys. Chem. (1996) 100, 7212; 0.1. Mićić, et al., J. Phys. Chem. (1995) 99, 77549). TOPO also has a high boiling point and is a stable compound at reaction temperatures below 380° C. (Guzelian, 1996; Mićić, 1995). Similar to its role in CdSe (Murray, 1993) and InP nanoparticles (Guzelian, 1996; Mićić, 1995), TOPO may coordinate surface gallium and other Group III acceptor sites, providing a passivating shell to terminate growth and also preventing agglomeration among particles.

In one embodiment, the nanoparticles are GaN or GaInN. For example, gallium chloride (GaCl₃) and triethylamine ((C₂H₅)₃N) may be used as the source materials, trioctylphosphine oxide (TOPO) as the capping agent and acetonitrile (CH₃CN) as the solvent. Alternative source materials include but are not limited to gallium bromide, gallium iodide, indium chloride, indium bromide, indium iodide, aluminum chloride, aluminum bromide, aluminum iodide, boron chloride, boron bromide, boron iodide, gallium dimethylamine Ga₂(N(CH₃)₂)₆, gallium diethylamine Ga₂(N(C₂H₅)₂)₆, trimethylamine, trimethylphosphine (CH₃)₃P, triethylphosphine (C₂H₅)₃P, triethylenediamine (C₆H₁₂N₂), triethylarsine (C₂H₅)₃As, trimethylarsine (CH₃)₃As, trimethylantimony (CH₃)₃Sb, and triethylantimony (C₂H₅)₃Sb. Dialkyl and monoalkyl compounds of the Group V element, including, for example, 1, 2, 3, 4, 5, or more carbons, may also be used. Mono-, di-, and tri-alkyl compounds of the Group V element may also be chelated with the Group III element to form the source material. The Group V source material may be optimized for the lability of the Group V atom and the boiling point of the material. In one embodiment, triethylamine may be used as both reagent and solvent, without adding acetonitrile as a solvent. Alternative solvents include but are not limited to acetonitrile, chloroform, benzene, paraffin oil, and naphthalene. Alternate borate salts, such as sodium tetrahydroborate (the terms tetrahydroborate and borohydride are used interchangeable herein), may also be used.

Depending on the source materials and the particular Group III and Group V element, it may be desired to use alternative reducing agents, e.g., lithium aluminum hydride, lithium triethylborohydride, lithium trimethoxyaluminum hydride, etc. One skilled in the art will be familiar with a variety of reducing agents. Exemplary reducing agents are described in Carey, et al., Advanced Organic Chemistry, Plenum Press, 1990, Third Edition, the contents of which are incorporated herein by reference. Without being bound by any particular theory, we propose that an exemplary basic reaction chemistry follows:

GaCl₃+(C₂H₅)₃N+3KBH₄→GaN+3KCl+3C₂H₆+3BH₃+1.5H₂ (1)

While the reaction can proceed at room temperature, synthesis at temperatures between 180 and 450° C. resulted in a mixture of amorphous and crystalline material, where the crystallized materials were of 1 to 4 nm size, as observed by high-resolution TEM and XRD observations. Lower temperatures, e.g., down to about 100° C., may also be employed, depending on the degree of crystallinity desired. Sub-nanometer sized clusters were also observed. To more fully crystallize the product, the reaction products may be condensed to a viscous colloidal gel by vaporization of the solvent, and then placed in a sealed steel vessel and heated at temperatures ranging from 300 to 450° C. for 8-16 hours. The resulting material is then purified to extract the III-V nanoparticles, resulting in substantially spherical particles ranging in diameter from about 4 to about 15 nm. Increased reaction times and temperatures increase the size of the particles and may be used to increase the particle size to 20 nm, 30 nm, or even larger. Increased reaction times also reduce polydispersity. In some embodiments, the size variation of the particles is less than 15%, less than 10%, or less than 5%. The various reaction products may be removed by partitioning them into various solvents or simply washing the nanoparticles (See Examples).

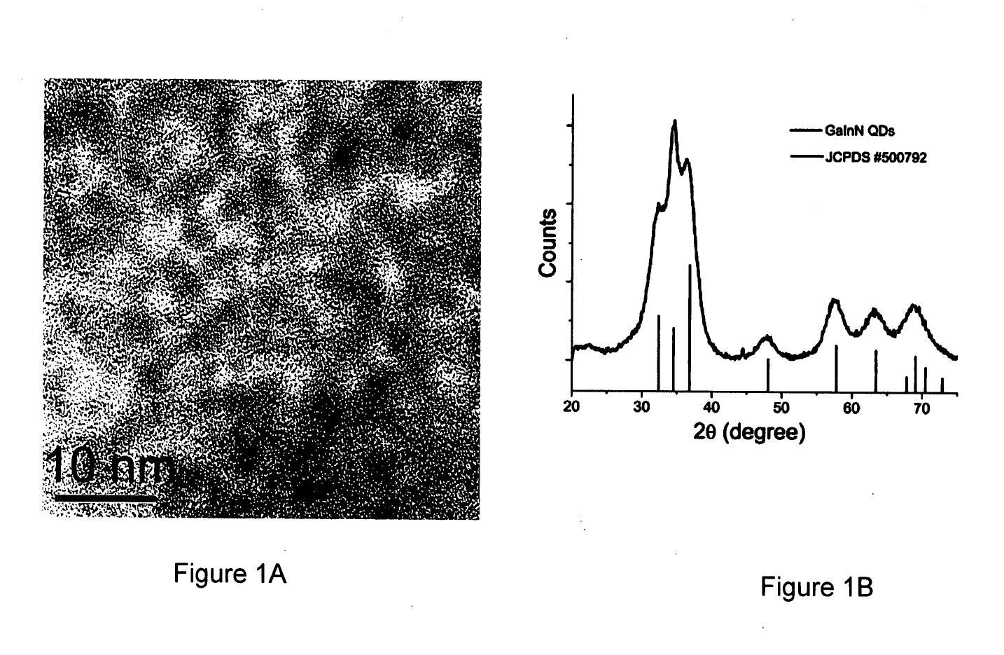

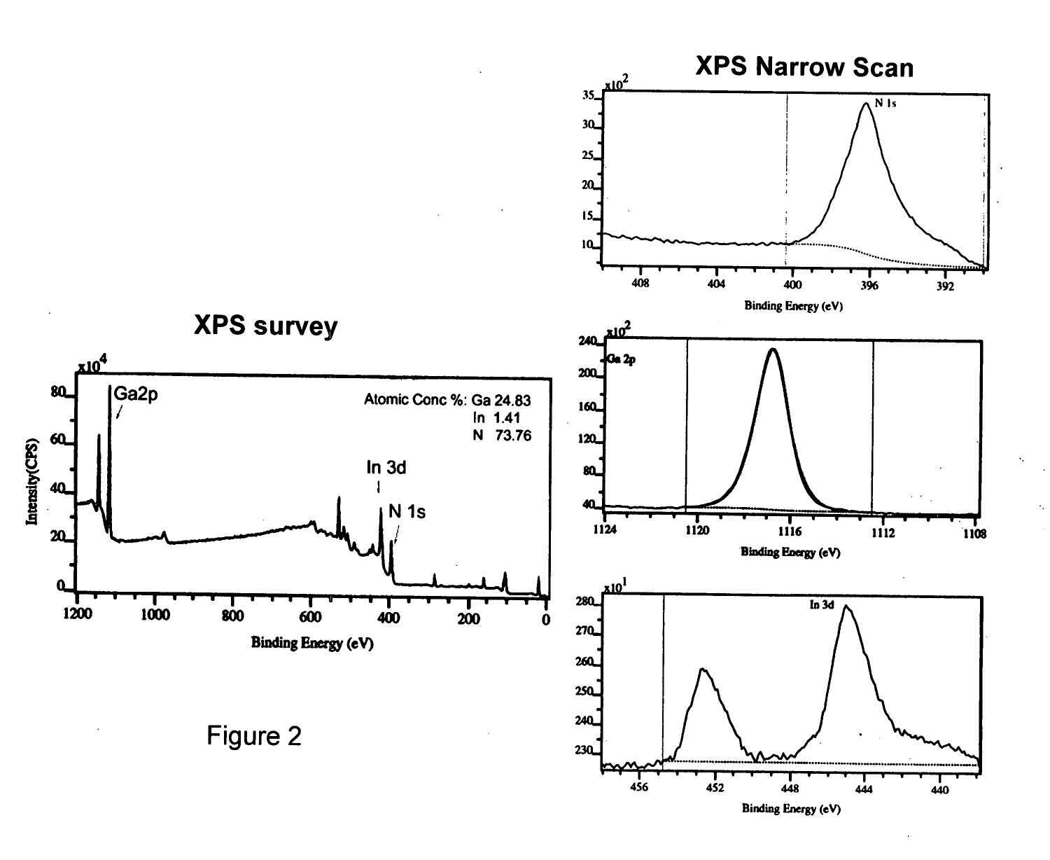

One skilled in the art will recognize that these methods may be extended to produce other III-V materials. For example, triethylphosphine may be substituted for triethylamine. Other trichloride, trihalide, or other salts may be substituted for or combined with gallium chloride to form binary or mixed III-V materials. For example, the techniques of the invention may be used to produce nanoparticles of BN, BP, ALP, AlAs, AlSb, AlN, GaP, GaAs, GaSb, InP, InAs, InSb, InN, AlGaN, AlGaP, AlGaAs, GaInAs, GaInN (GaInN and InGaN are used interchangeably without consideration for the relative ratios of Ga and In), GaInP, and other III-V materials combining two or more of Al, Ga, and In with N, P, As, or Sb. An XRD pattern and TEM image of Ga0.95In0.05N nanoparticles is shown in FIG. 1; an XPS survey is shown in FIG. 2. III-V materials may be produced with any mixtures of Group III elements in any proportion between 1:99 and 99:1, for example, 95:5, 90:10, 80:20, 70:30, 60:40, 50:50, etc. (and, of course, the reverse compositions, 5:95, 10:90, etc., depending on which Group III element is “first”) The only limit on the proportion is effect of lattice mismatch between the Group III materials, which may lead to precipitation of a non-mixed III-V material. III-V compounds doped with 0.1 to 10% of transition metals or rare earth metals, e.g., Mn, Co, and Eu, may also be produced using the teachings of the invention. In one embodiment, the transition metal is magnetic. For example, adding EuCl₃ to the reagents listed in formula (1) allows formation of GaN:Eu. Halides, organometallic chelates (e.g., with mono-, di-, or tri-alkyl compounds of the Group V element), or other salts of rare earth or transition metals may be used to produce doped binary or mixed III-V materials. One skilled in the art will recognize that the absorption and emission wavelengths of nanoparticles may be easily adjusted by changing the composition or size of the particles, by changing the composition of the capping agent, or by adjusting the ratios of the group III materials in mixed III-V nanoparticles.

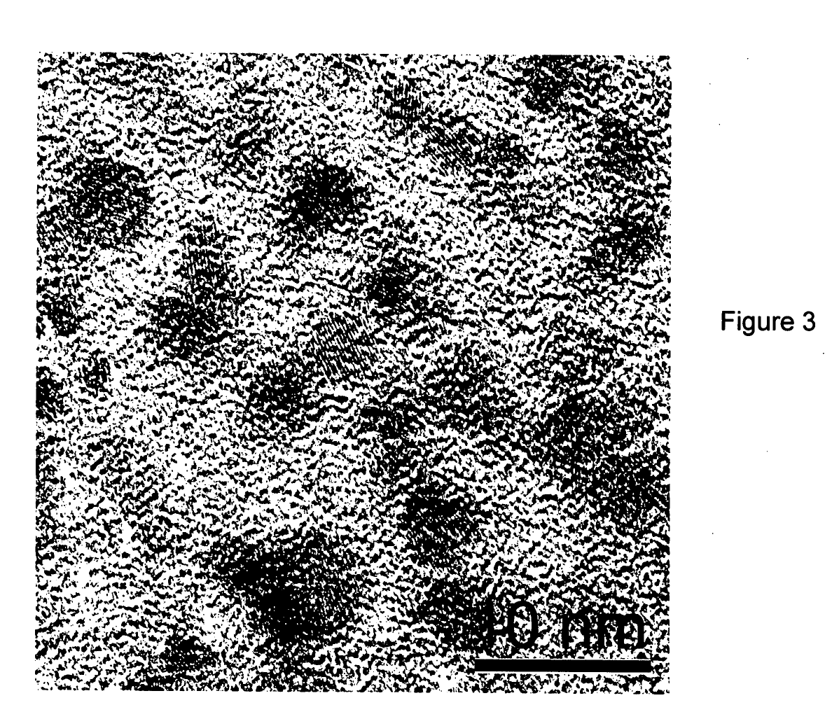

In another embodiment, combinations of III-V materials may be formed in core shell structures. For example, the III-V nanoparticles produced according to an embodiment of the invention or any other method and having an aminated surface (e.g., after evaporation from a solution in diethylamine) may be suspended in a solution of a salt of the desired Group III element with added chloroform and pre-heated to about 200-350° C., following which a source material for the Group V compound is added to the solution. Where purified particles are used as the core, the core shell structures may be added to a solution containing source materials for the Group III and Group V and prepared in the same manner as the “core” structures described above. In one embodiment, the group V element is the same in both the core and the shell materials. In another embodiment, the group III element or elements are the same. In another embodiment, both the group V and the group III elements are different between the core and the shell. A TEM image of (InN)GaN core shell nanoparticles (as described herein, the species in parentheses forms the core) is shown in FIG. 3. Core shell structures may be used to further tune the emission spectra of III-V nanoparticles. The thickness of the shell should be sufficient to provide good contact between the shell and the core without any delamination resulting from lattice mismatch between the core and shell materials. In some embodiments, the shell thickness may range from about 0.5 to about 2.5 nm, for example, about 1 nm, about 1.5 nm, or about 2 nm. Alternatively, or in addition, the ratio of the shell thickness and the core radius may be between about 1:1 and 1:5, e.g., about 1:2, about 1:3, or 1:4.

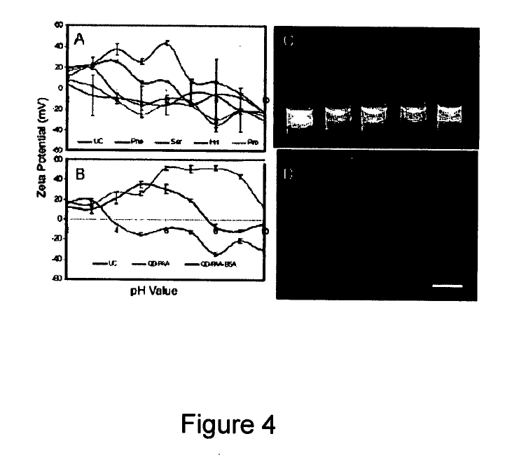

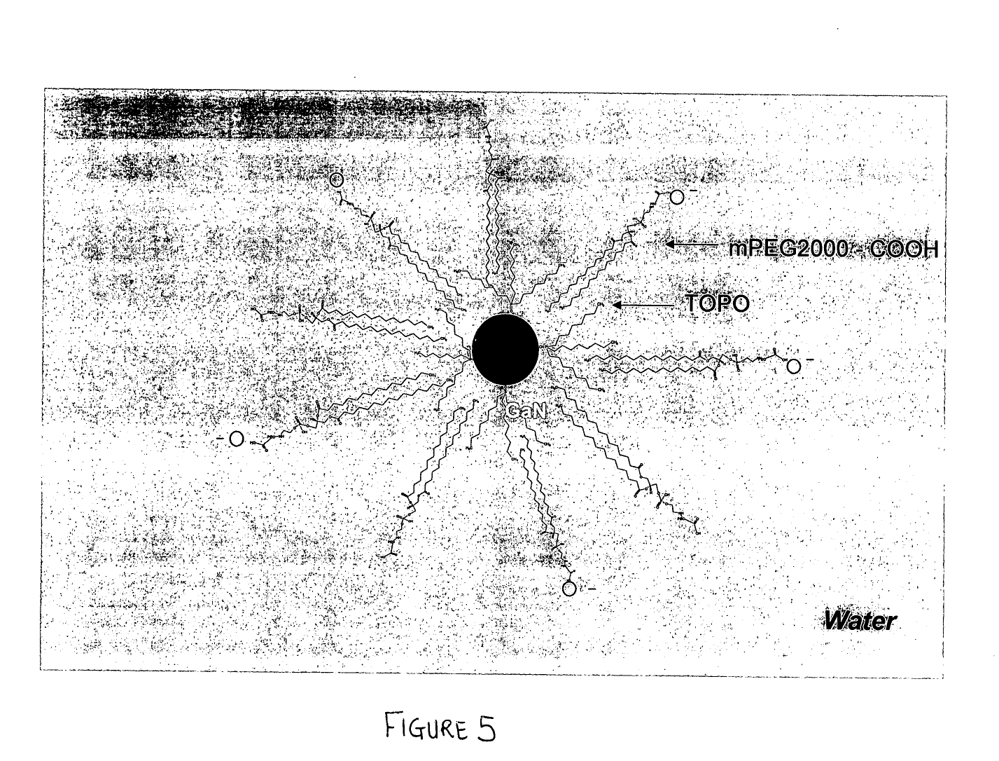

It is widely appreciated that the nature of the capping material of colloidal semiconductor nanoparticles exerts a strong influence on their optical quality. More recently, ‘bio-functionalized’ capping of nanoparticles allows their utilization as efficient, non-bleaching fluorophores for in vivo tagging of cells (X. Gao, et al., Nat. Mater. (2004) 22, 969). Finally, control of the net charge of nanoparticles through strategic control of the capping material has allowed selective deposition of nanoparticles through electrostatic affinities (S. Jaffar, et al., Nano Lett (2004) 4, 1421). In one embodiment, negatively or positively charged capping layers may be employed. In one example, the surface-capping TOPO was removed from GaN nanoparticles and alternative capping layers were applied. The zeta potential of bare GaN nanoparticles is shown in FIGS. 4A and 1B and appears predominantly negative, with the isoelectric point between pH 3 to 4. Without being bound by any particular theory, we propose that the high negative charge may be due to the presence of hydroxyl molecules attached to the dangling gallium ions, as observed for other metal nanoparticles (Jaffar, 2004). Capping the nanoparticles in cationic polyallylamine (PAA) resulted in a shift to positive values, as shown in FIG. 4B. This charge reversal may be attributable to excess deposition of PAA on GaN nanoparticle surfaces that caused overcompensation of charge neutralization. The cationic PAA-coated GaN nanoparticles were deposited on glass substrates stamped with negatively charged hyaluronic acid (HA), similar to the procedure described in reference (Jaffar, 2004), the contents of which are incorporated herein by reference, producing the line patterns shown in FIG. 4D. The blue fluorescence is caused by the positively charged PAA-GaN nanoparticles bound to HA patterns on glass, and the dark areas are the bare, GaN non-binding regions, of the glass surface. The nanoparticles are deposited in regular, well defined patterns with sharp edges and high fidelity. This suggests that the nanoparticles interact specifically with the charged adhesive substrate, with minimal non-specific adsorption. The electrostatic interactions between the nanoparticles and the substrate are strong enough to withstand repeated rinsing, and the patterns do not distort or aggregate even after drying. Furthermore, the patterns are reproducible over large areas and with varying feature size and shape (data not shown).

Other organic agents, including those that are used as capping agents for quantum dots, may also be employed as capping agents for the III-V nanoparticles. Exemplary capping agents include acetamidine hydrochloride, cetyltrimethyl ammonium bromide, and benzalkonium chloride. Exemplary agents have an end group that can bind to the nanoparticle, such as chemical groups that include S, P, O, or N. Exemplary groups include thiol, phosphines, phosphine oxides, amine, amine oxides, sulfonates, carbonates, and carboxylates. These groups may anchor a variety of organic groups to the nanoparticle. In some embodiments, the group includes a hydrocarbon chain terminated by a reactive end group. The hydrocarbon chain may be a straight or branched alkane and may include electron rich groups such as amide, ketone, ether, or aromatics. Such groups may be included in the hydrocarbon chain or pendant from it. The reactive end group may include hydroxyl, amine, amide, carboxylate, sulfonate, phosphate, ammonium, etc. Alternatively or in addition, polyelectrolytes may be employed as capping agents. Exemplary polyelectrolytes include, in addition to PAA and HA, poly(vinylsulfonic acid), linear and branched poly(ethylene imine) PEI, polyallylamine HCl (PAH), polylysine, chitosan, poly(diallydimethylammonium chloride) (PDAC), polysaccharides, polymers of positively charged amino acids, polyaminoserinate, hyaluronan, polymalic acid, polyimides, polymers of negatively charged and acidic amino acids, and polynucleotides.

An innovative approach to determining effective capping agents for the nanoparticles utilized biological based peptide selection against bare GaN nanoparticles by using a combinatorial library of genetically engineered M13 bacteriophage viruses (S. R. Whaley, et al., Nature (2000) 405, 665; C. E. Flynn, et al., Acta Mater. (2003) 51, 5867, the contents of both of which are incorporated herein by reference). A 12 amino acid linear library of modifications to the p3 peptide on M13 was used to identify peptide binding motifs for GaN. Several successful binding peptides from the screening of the linear library on GaN were isolated. After three rounds of selection, several dominant binding motifs emerged and were termed G8 and G9, with the amino acid sequences (Ser-Ser-Phe-Ser-Asn-Val-Thr-Ser-Gly-Thr-Gln-Lys) and (Lys-Leu-His-His-Ser-Pro-Pro-Pro-Pro-Phe-Val-Phe), respectively. A third binding motif, Val-Ser-Pro-Ser-Gly-Thr-Pro-Glu, was also identified by biopanning against a library of p8 modified M13. The peptides expressed on the virus were tested and confirmed to have binding specificity to GaN crystal surfaces. The binding of four of the component amino acids from peptide G9, phenylalanine (Phe), histidine (His), serine (Ser) and proline (Pro), to the GaN nanoparticle surfaces was subsequently individually assessed. The surface charge of the GaN particles capped with the various amino acids is shown in FIG. 4A. The presence of histidine and proline surface ligands does not significantly alter the pH response of the nanoparticle surface charge. However, phenylalanine and serine residues render the nanoparticle surfaces less negative and increase the isoelectric points to approximately pH 6.5 and 9, respectively. This may be attributed to the basic nature of the side chains, which have pKa's of 9.2 and 13, respectively. Therefore, the surface charge of the nanoparticle may be affected by the chemical properties of the surface ligand. FIG. 4C shows vials of equal concentrations of GaN nanoparticles capped with TOPO, His, Phe, Pro or Ser. The vials were illuminated by a UV lamp, and show some variations in fluorescence output. One skilled in the art will recognize that the same techniques may be used to identify appropriate amino acid capping agents for other nanoparticle compositions.

Additional functionalities may be attached to these nanoparticles that may enable specific cellular targeting and promote specific interactions within cells. Since the nanoparticle cores are not cytotoxic, such bioconjugated nanoparticles coupled with proteins, peptides, antibodies, or other ligands may prove to be effective cellular probes. The successful demonstration that larger proteins such as bovine serum albumin can be coupled to the PAA-GaN nanoparticles (FIG. 4B) suggests that antibodies and other ligands can be complexed with these nanoparticles and used for specific cellular targets. In one embodiment, the desired functionality is directly attached to the nanoparticle as a capping agent. Alternatively or in addition, the desired functionality is attached to a reactive group on the capping agent. For example, a biologically active agent may be linked to an amine, carboxylate, thiol, or other reactive group on a capping agent via carbodiimide chemistry (e.g., using NDC or similar reagents) or other coupling reactions. Hexahistidine oligomers or biotin may also be directly attached to the particles or retained on a capping layer. In this embodiment, a biologically active agent derivatized with streptavidin may be used to conjugate the biologically active agent to the nanoparticle. In another embodiment, a nucleic acid oligomer is attached to the nanoparticle, while an oligomer having an at least partially complementary sequence, e.g., at least 70%, at least 80%, at least 90%, or at least 95% complementary, is attached to the desired biologically active agent. Biologically active agents may also be non-covalently retained on a capping layer. For example, a positively charged agent may be bound to a negatively charged group in a capping agent through electrostatic interactions or ionic bonds. Additional non-covalent interactions by which materials may be retained on a nanoparticle via a capping agent include van der Waals interactions, hydrogen bonding, magnetic interactions, ligand-receptor interactions, and π orbital-bonding. Individual amino acids or polypeptides may be linked directly to nanoparticles or to the capping agent via covalent or non-covalent interactions.

The small size, bright fluorescence and aqueous stability of the III-V nanoparticles make them good candidates for intracellular fluorescent tags in biological applications, In addition, since the use of gallium for medical imaging (E. Even-Sapir, et al., Eur J Nucl Med Mol Imaging (2003) 30 (Suppl. 1), S65; W. Becker, et al., Lancet Infect Dis. (2001) 1, 326), as an anti-cancer therapeutic (C. R. Chitambar, Current Opinion in Oncology. (2004) 16, 547), and as a drug for increasing bone density is already approved by the FDA, potential therapeutic applications using GaN nanoparticles may be more feasible than II-VI semiconductor nanoparticles that have been shown to cause cytotoxicity (A. M. Derfus, et al., Nano Letters (2004) 4, 11). GaN nanoparticles may exhibit less of the oxidation and cytotoxicity associated with II-VI nanoparticles.

In one embodiment, a targeting agent may be covalently or non-covalently linked to the nanoparticles. Once in an in vivo or in vitro environment, cells that have receptors that are sensitive to the targeting agent used will take up the targeting agent-nanoparticle conjugates. Targeting agents may include but are not limited to antibodies and antibody fragments, nucleic acid ligands (e.g., aptamers), oligonucleotides, oligopeptides, polysaccharides, low-density lipoproteins (LDLs), folate, transferrin, asialycoproteins, gp120 envelope protein of the human immunodeficiency virus (HIV), carbohydrates, polysaccharides, enzymatic receptor ligands, sialic acid, glycoprotein, lipid, small molecule, bioactive agent, biomolecule, immunoreactive fragments such as the Fab, Fab′, or F(ab′)₂ fragments, etc. A variety of targeting agents that direct pharmaceutical compositions to particular cells are known in the art (see, for example, Cotton, et al., Methods Enzym. 217:618; 1993; incorporated herein by reference). Targeting agents may include any small molecule, bioactive agent, or biomolecule, natural or synthetic, that binds specifically to a cell surface receptor, protein or glycoprotein found at the surface of cells.

Particular capping agents may also be provided for the nanoparticles to specifically render them water soluble. Exemplary coatings include organic phosphates, for example, phospholipids, phosphocholines or PEG-derivitized phospholipids or phosphocholines. Exemplary phospholipids include 1,2-dipalmitoyl-sn-glycero-3-phosphoethanolamine-N-[methoxy(polyethylene glycol)-2000] (ammonium salt) (DPGP-PEGm), 1,2-distearoyl-sn-glycero-3-phosphoethanolamine-N-[carboxy(polyethylene glycol)2000] (ammonium salt) (DPGP-PEGc), and 1,2-dipalmitoyl-sn-glycero-2-phosphocholine (DPPC). Alternatively or in addition. PEG of different molecular weights or with different end groups (e.g., amine) may be employed. In one embodiment, an organic solvent in which the nanoparticles are soluble is combined with the capping agent at room temperature. III-V nanoparticles are added to the solution and the solvent evaporated slowly, e.g., overnight. Water is added to the dried product and heated, for example, to between 50° and 90° C., for example, to about 60°, about 70°, or about 80°. Optionally, the resulting solution may be dialyzed against 18 Mohm water to remove excess reagents. Suitable dialysis membranes will be familiar to those of skill in the art. An exemplary water soluble GaN nanoparticle is shown in FIG. 5.

The cells can be identified by exposing them to light having an energy at which the nanoparticle luminesces. Different types of cells may be identified concurrently by labeling nanoparticles with different band gaps with targeting agents corresponding to receptors that are unique to the cell that is being identified. In one embodiment, the nanoparticles may be used to label cells for flow cytometry. Alternatively or in addition, the nanoparticles may be used to label cells or tissue for fluorescence microscopy. For example, the nanoparticles may be used to identify a particular group of cells in a larger population or to label cells in vivo or in vitro for later microscopic examination.

Control of the capping material enables control of the net surface charge of the nanoparticles. This in turn allows selective deposition of nanoparticles through electrostatic interactions with a substrate, allowing nanoparticles to be deposited on selected regions. Substrates may patterned using any technique known to those of skill in the art, for example, the methods disclosed in U.S. Pat. No. 6,180,239 by Whitesides, C. S. Dulcey et al., Science, 1991, vol. 252, pp. 551-554, and Dulcey, et al., Langmuir, 1996, 12 (6), 1638-1650. For example, hyaluronic acid or other negatively charged material may be patterned on a substrate, following which nanoparticles capped with a positively charged material, e.g., PAA, will preferentially deposit on the HA layer with respect to the uncoated glass. Other materials that may be patterned on a surface to selectively adjust surface charge include but are not limited to any of the polymers described as potential capping agents, SAM forming molecules having variously charged or chargable endgroups. For example, PDMS stamps may be used to deposit materials onto a substrate. PDMS stamps may be used to deposit materials onto a substrate. Alternatively, materials having a natural negative charge may be patterned with a positively charged material. In another embodiment, plasma etching may be used to create negatively charged regions on a surface. Standard lithographic techniques may be used to protect desired regions of the substrate from being etched. Of course, these techniques may also be used to prepare cast (e.g., unpatterned) films of nanoparticles on substrates such as polymers, metals, ceramics, and semiconductors.

Nanoparticles may be formed into biofilms. For example, M13 or Fd bacteriophages may be used as scaffolds to organize nanoparticles based on biomolecular recognition and self-assembly. The particular phage clones that are appropriate for a given nanoparticle composition may be selected through biopanning as discussed above. To form phage-nanoparticle hybrid films, the desired phage clone may be suspended in a solution of the nanoparticle. At sufficiently high concentrations, the hybrids form liquid crystals that can be cast into films (see Lee, et al., Science, 2002, 296:892 and Lee, et al., Langmuir, 2003, 19: 1592, the contents of both of which are incorporated herein by reference). The viral/nanoparticle conjugates may be assembled into films and nanowires using the techniques disclosed in using the techniques described in U.S. Patent Publication No. 20040171139 and Lee, et al., Adv. Mater., 2003, 1:689, the contents of both of which are incorporated herein by reference. In one embodiment, GaN nanoparticles were bound to pIII fusions with the sequences Tyr-Pro-Thr-His-His-Ala-His-Thr-Thr-Pro-Val-Arg, Thr-Ser-Asp-Ile-Lys-Ser-Arg-Ser-Pro-His-His-Arg, and Lys-Leu-His-His-Ser-Pro-Pro-Pro-Pro-Phe-Val-Phe and to pVIII fusions with the sequence Val-Ser-Pro-Ser-Gly-Thr-Pro-Glu.

Nanoparticles may also be pressed into films. For example, nanoparticles of one composition or mixtures with various compositions (either random or in some geometric pattern) may be pressed using hot isostatic pressure (“hipping”) at about 20V-400° C. and, for example, 1000 psi for at least 8 hours. Hipping is well known to those of skill in the art and is described in Atkinson, Metallurgical and Materials Transactions A, 2000, 31A: 2981, the contents of which are incorporated herein by reference. Standard powder processing techniques may be used to vary the processing temperature, pressure, and time to achieve different final shapes and sizes. Hipping provides substantially void free films of 1 mm or greater thickness and substantially uniform density.

In an alternative embodiment, particles may be cast into solids macrostructures using the techniques described in Mao, et al., Adv. Funct. Mater., 2003, 13: 648, the contents of which are incorporated herein by reference. Briefly, a solution of nanoparticles, e.g., about 1 mM, is heated well above room temperature, e.g., about 50°-90° C., and then added dropwise to a solvent that has been cooled to well below room temperature, e.g., about −25° C. to about 0° C. The difference in temperatures, the capping agent, and the concentration of the solution may be adjusted to adjust the arrangement and shape of the resulting quantum dot solids. After leaving the solution at the low temperature for about 12-24 hours, the solution may be brought to room temperature. Other techniques for preparing assemblies of nanoparticles known to those skilled in the art may also be employed.

The nanoparticle-containing films may be employed in a variety of optical, electrical, and biological applications. For example, films may be used to produce optical waveguides, light amplifiers, optical displays, photovoltaic devices, biosensors, and other devices. Examples of some of these are discussed below.

In one embodiment, nanoparticle-containing films may be used as optical waveguides. These waveguides may be used to direct a timing pulse to various parts of a semiconductor chip, helping to synchronize various functions on the chip. Alternatively, waveguides may be used to transfer data from one point to another on a chip or circuit board or from one circuit board to another. For example, a laser diode may be used to transform an electrical signal into an optical signal. The optical signal is transferred across the waveguide to a photodiode in a second location. The photodiode transforms the optical signal back into electrical bits. The waveguides are formed by disposing the nanoparticle-containing films between cladding layers. Silicon oxide is commonly used as a cladding layer because it can double as a passivation layer for circuitry already present on a silicon wafer. The second cladding layer may be an air gap, an additional layer of silica, or a polymer having a refractive index sufficiently different from that of the film.

Nanoparticle-containing films may also be employed as optical amplifiers. Incident blue light entering an optical amplifier containing a nanoparticle film excites electrons in the particles. The electrons relax through a radiative mechanism, causing light emission in the same wavelength as the incident, exciting radiation. Some of the emitted photons participate in the excitation and emission process, while others proceed through the film, adding to the intensity of the incident radiation. These amplifiers may be used in repeaters in fiber optic networks or to provide preamplification in optical receivers for high bit rate applications.

Nanoparticle-containing films may also be used in optical displays and photovoltaic devices. In both cases, the photoelectric effect is employed to turn an electrical voltage into a photon or vice versa. In optical displays, the film may be disposed over an grid of thin film transistors, just as an active matrix LCD screen is produced by disposing a liquid crystalline film over a grid. When a given thin film transistor is switched on, it directs electric current across a specific point in the film. The voltage excites electrons in the nanoparticles, which emit a photon as they relax. By combining nanoparticles that emit blue, green, and red light in one or more films, a full spectrum of colors may be generated. Photovoltaic displays work via the opposite mechanism. The nanoparticle-containing film may be disposed in a solar cell. Sunlight incident on the film excites electrons to an energy level where they may be conducted, essentially generating a flow of electricity.

III-V nanoparticles may also be used in any application where quantum dots or nanoparticles have been employed. For example, nanoparticles have been investigated for use in LEDs (see Tanaka, et al., Review of Laser Engineering, 2004, 32: 410-413; Passaseo, Appl. Phys. Lett., 2003, 82: 1818), microelectronics (see Li, et al., Science, 2003, 301:809), environmental lighting (e.g., buildings and cars) (see Achermann, et al., Nature, 2004, 429: 642-646), lasers (see Sellers, et al., Physica E, 2005, 26: 382-385), and solar cells (see Schaller, et al., Phys. Rev. Lett., (2004) 92:186601).

### EXAMPLES

### GaN Nanoparticle Synthesis—Method 1

Gallium chloride, triethylamine, trioctylphosphine oxide (TOPO) and acetonitrile were obtained from either Alfa-Acer or Sigma-Aldrich, and used as received.

In a glove box under a nitrogen atmosphere, 1.76 g (10 mmol) GaCl₃, 1.62 g (30 mmol) KBH₄, 10 mL CH₃CN, 5 mL (36 mmol) (C₂H₅)₃N and 23 mg TOPO were consecutively added to a round bottom glass vessel while the solution was stirred. In an alternative embodiment, the acetonitrile may be omitted and 15 mL of triethylamine used. After the solution was stirred for 30 minutes, the vessel was sealed and removed from the glove box, and the solution was heated at 200° C. for 8 hours. After the solution was allowed to cool to room temperature, the cover of the glass vessel was loosened, and the vessel placed in a vacuum chamber to concentrate the product through evaporation of the solvent. A portion of the remaining solids was placed in a steel pressure vessel and heated at 350° C.

### GaN Nanoparticle Synthesis—Method 2

GaN nanoparticles were prepared according to method 1, but without TOPO.

### GaN Nanoparticle Synthesis—Method 3

GaN nanoparticles were prepared according to method 1, but substituting acetonitrile for chloroform.

### GaN Nanoparticle Synthesis—Method 4

5 mL of the concentrated product of Method 1 was placed into a steel high pressure vessel together with 1-2 mL of triethylamine and heated at 350-450° C. for 10 hours to improve crystallization. Particles produced according to these methods had diameters between about 2 and about 10 nm.

### InN Nanoparticle Synthesis

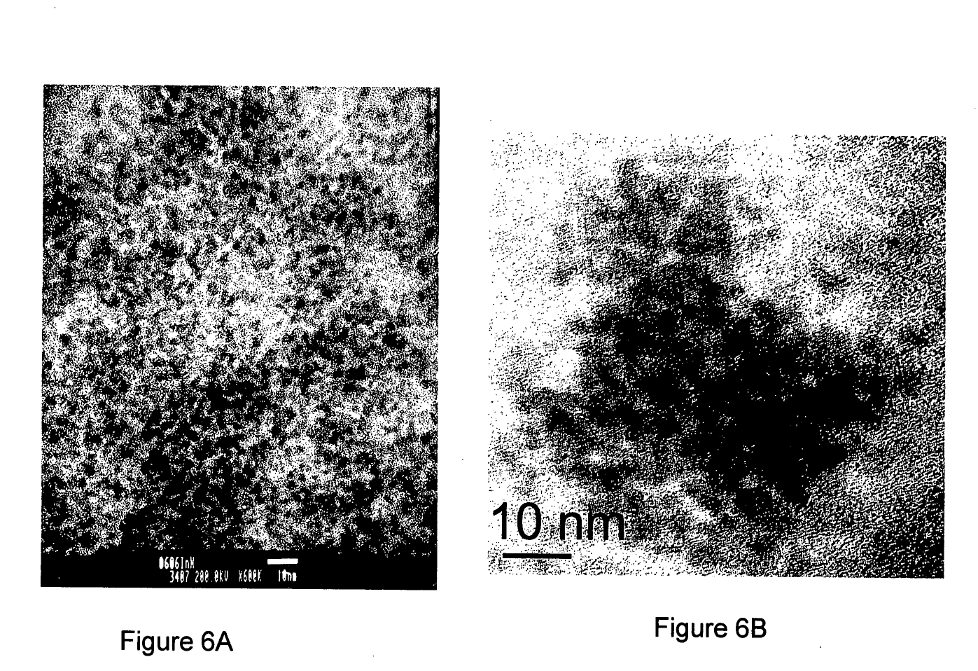

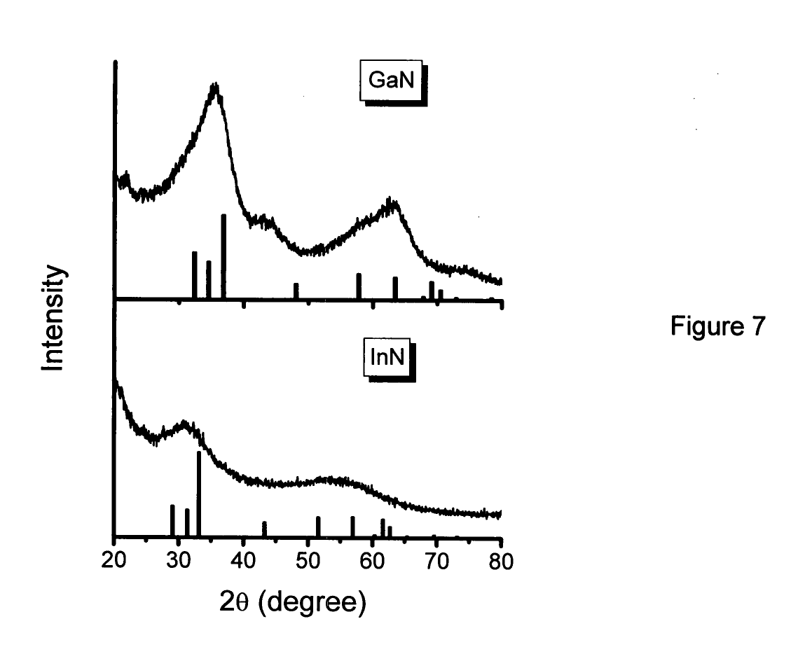

Five grams of TOPO was dissolved in 10 mL chloroform in a round bottom flask. After five minutes of stirring, 1.11 g (5 mmol) indium chloride and 0.81 g (15 mmol) potassium borohydride are added to the solution, following which 5 mL (36 mmol) of triethylamine was slowly added while stirring. After 30 minutes of stirring, the flask was sealed and the solution heated at 200-450° C. for 12 hours. The solution was allowed to cool to room temperature, following which the dark brown colored InN suspension could be purified. In other synthetic methods, the TOPO was omitted and/or acetonitrile was substituted for chloroform. The resulting InN particles were about 5 nm in diameter and are shown in FIG. 6. XRD patterns of the GaN and InN nanoparticles are shown in FIG. 7.

### AlN Nanoparticle Synthesis—Method 1

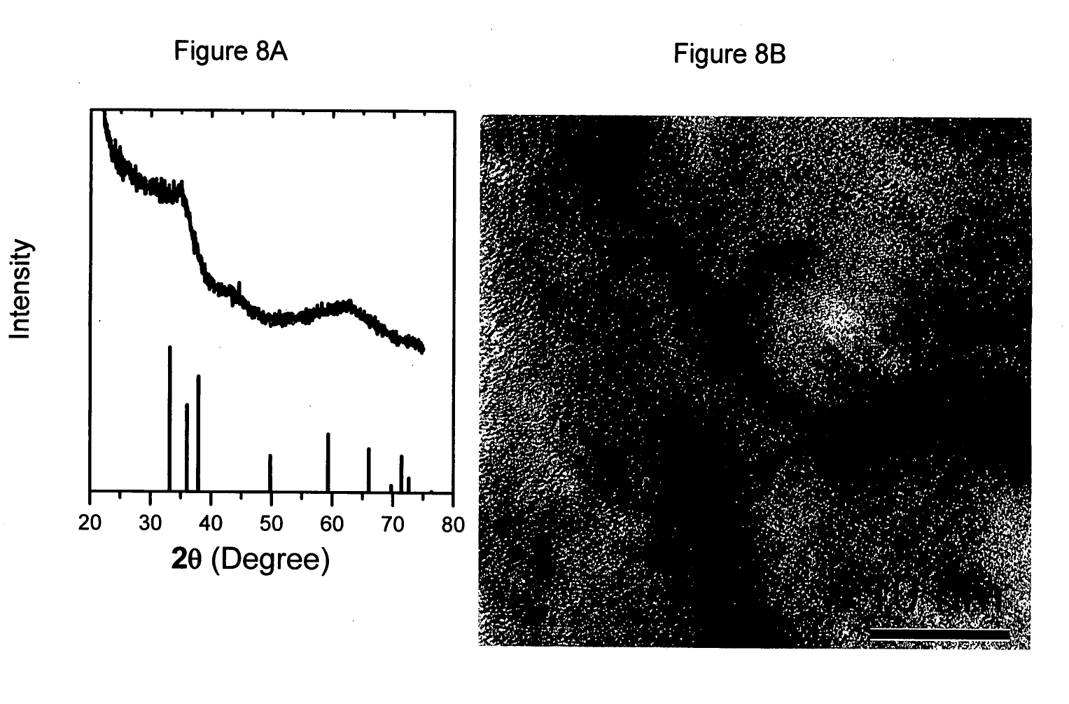

15 mL (108 mmol) of triethylamine was slowly added to 6.667 g (50 mmol) aluminum chloride in a round bottom flask while stirring. After 30 minutes of stirring, the flask was sealed and the solution heated at 200-450° C. for 12 hours. The solution was allowed to cool to room temperature, and the yellow-brown colored AlN suspension was purified. An XRD pattern and image of the nanoparticles, which were about 3 nm in diameter, is shown in FIG. 8. In an alternative embodiment, 1.62 g (30 mmol) potassium borohydride was added to the solution while stirring, following which the flask was sealed and heated as above.

### AlN Nanoparticle Synthesis—Method 2

2.6 g (25 mmol) triethylaluminum and 10 mL triethylamine were stirred in a round bottom flask for 30 minutes. The flask was sealed and the solution heated at 200-450° C. for 12 hours.

### GaInN Nanoparticle Synthesis.

GaInN nanoparticle synthesis is similar to that for synthesis of GaN nanoparticles. 1.76 g (10 mmol) GaCl₃, 0.44 (2 mmol) InCl₃, 1.62 g (30 mmol) KBH₄, 10 mL CH₃CN, 5 mL (36 mmol) (C₂H₅)₃N and 30 mg TOPO were consecutively added in a round bottom glass vessel while the solution was stirred. After the solution was stirred for 30 minutes, the vessel was sealed and the solution was heated at 200° C. for 12 hours. After the solution cooled down to room temperature, the cover of the glass vessel was loosened, and the vessel placed in a vacuum chamber to concentrate the dark brown colored suspension through evaporation of the solvent. A portion of the remaining material was placed in a steel pressure vessel and heated at 350° C. for 16 hours.

### GaN Nanoparticle Purification

The purification process utilized differences in the weights and solubilities of the products. For example, to remove the KCl, the product solution was dissolved in a 1:1 (v/v) mixture of glycerol and ethanol and stirred for 30 minutes. The solution was allowed to settle for 60 minutes. The top of the solution (80%) was carefully removed by a pipette and spun at 14000 rpm for 15 minutes, causing the GaN nanoparticles to deposit at the bottom of the centrifuge tube. To remove the hydrophobic organic molecules, mainly TOPO, the purified products were dispersed into acetone and sonicated at 80° C. for 15 minutes, and then centrifuged at 14000 rpm for 15 minutes. The supernatant containing the dissolved organics was discarded, leaving flocculates of the purified sample. This procedure was repeated several times to increase the purity of the sample. The sample was finally dried under vacuum, leaving concentrated, high purity nanoparticles. Nanoparticles were stored in sealed glass bottles at room temperature.

### Analysis of GaN and GaInN Nanoparticles

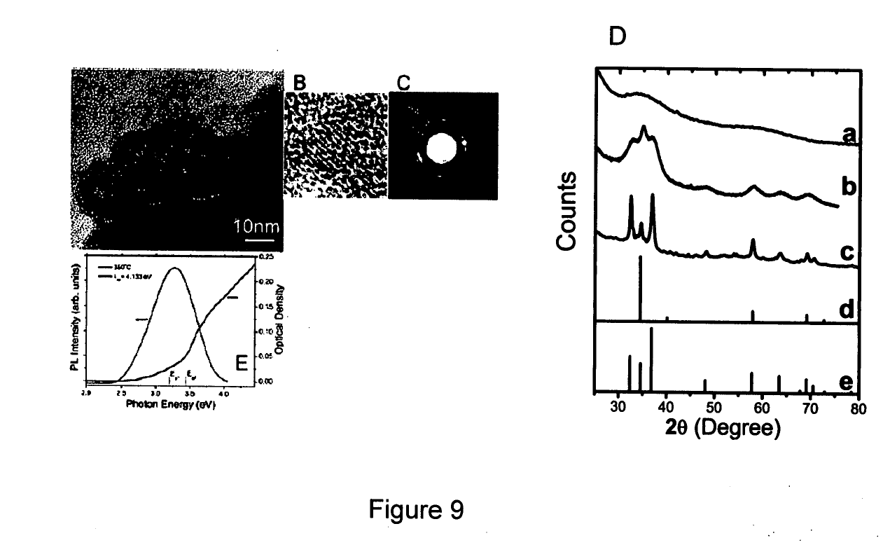

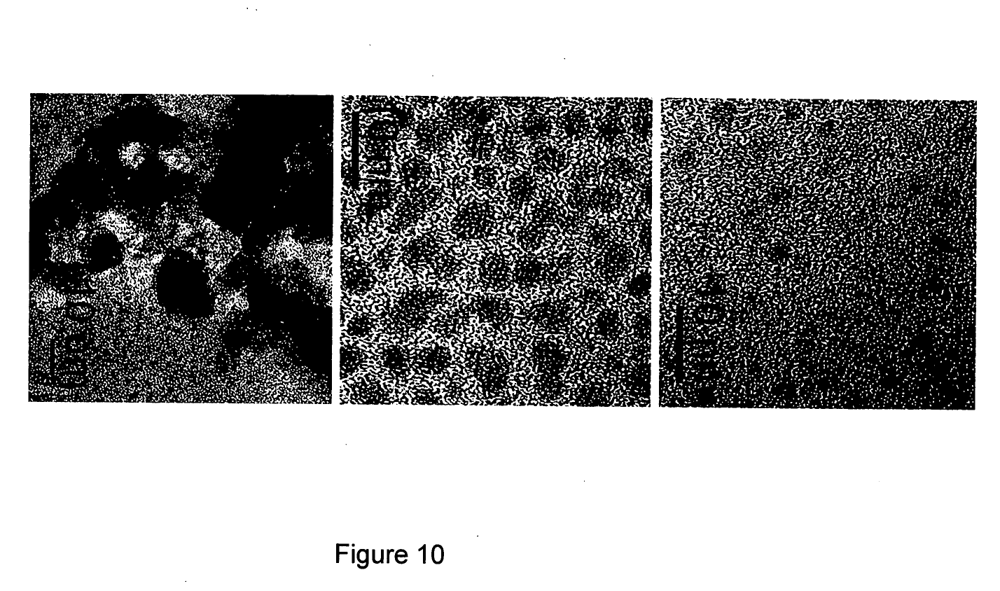

TEM images were obtained using a JEOL 2000 and 2010F at an accelerating voltage of 200 kV. FIG. 9A shows a bright field TEM image of GaN nanoparticles that were processed at 350° C.; the GaN particle sizes range from 2.7 nm to 6 nm in diameter. FIG. 9B provides a high resolution TEM image of an individual GaN nanoparticle and the corresponding electron diffraction pattern is shown in FIG. 9C. Lattice fringes were used to deduce spacings of 2.76 Å between crystal planes, consistent with the distances between the {100} planes of the hexagonal (wurtzite) structure of bulk GaN crystal. The electron diffraction patterns confirm the wurtzite structure as does the XRD data of FIG. 9D. The evolution of GaN nanoparticle size and orientation can be qualitatively monitored by comparing the XRD data for samples processed at increasing temperatures. The broad and relatively featureless XRD peaks for the 200° C. sample suggest both smaller average nanoparticle size and a distribution of orientations. The better defined XRD spectrum of the 450° C.-processed sample indicates a growth in nanoparticle size and excellent correspondence with the wurtzite (9D(e)) rather than zinc-blende (9D(d)) structure. FIG. 10 shows TEM images of a sample of GaN nanoparticles produced at 200°, 350°, and 450° C.

### Optical Absorption and Luminescence of GaN and GaInN Nanoparticles

Optical absorption and room temperature photoluminescence (PL) measurements were used to assess the optical properties of the GaN nanoparticles in solution. FIG. 9E shows both the absorption spectrum and photoluminescence spectrum of GaN nanoparticles processed at 350° C. The absorption spectrum is fairly featureless, with some suggestion of a broad shoulder or peak, indicating the wide distribution of nanoparticle sizes. Although the absorption increases substantially for photon energies greater than E_{g2} (3.45 eV, J. F. Muth, et al., Appl. Phys. Lett. (1997) 71, 2572), corresponding to the bandgap of wurtzite GaN, there is evident absorption at lower energies, which may correspond to defect states occurring below bandgap. The photoluminescence, under excitation by 4.13 eV photons from a deuterium lamp, displays a broad peak, centered at 3.27 eV. The breadth of the peak (0.7 eV FWHM) indicates the large dispersion of nanoparticle sizes. The PL emission peak occurs at lower energy than the band-edge of wurtzite GaN; this may be related to the effect of the piezoelectric potential in the nanoparticle, leading to a strong internal electric field, and resulting Stark shift in the emission.

Initial measurements of quantum efficiency established that GaN nanoparticles exhibited emission efficiencies comparable to those of CdSe/CdS core-shell dots synthesized in our labs that emitted at 570 nm. The GaN emission efficiency endures over several weeks of measurement. For example, the nanoparticle samples that were used for cell tags had been stored for two months and redispersed in water prior to use. In addition, the GaN nanoparticles are stable with respect to oxidation. An aqueous dispersion of GaN nanoparticles in an open cuvette was continually illuminated by a 80 watt UV lamp for 40 hours. The luminescence intensity of GaN nanoparticles decreased by about a third, while the luminescence of a control sample of commercial CdSe quantum dots almost completely quenched.

We also compared the photoluminescence from GaN nanoparticles with that from UV laser dyes. We used two kinds of laser dyes having optical absorption and light emission wavelength in the same wavelength regime as GaN nanoparticles. 2,5-Diphenylfuran (DPF, C₁₆H₁₂O, 98%) and 2,5-Bis-(4-biphenylyl)-oxazol (BBO, C₂₇H₁₉NO, 99%) were obtained from Alfa Aesar and used as received without further purification. The dye molecules were diluted to 1 nM in dioxane, and the GaN nanoparticles were diluted to the same concentration 1 nM (as GaN molecules) in deionized water. All the solutions were transferred into quartz cuvettes, and fluorescence was measured at an excitation wavelength of 300 nm. The emission efficiency of the GaN nanoparticles is about 47% that of BBO.

### Production of GaN-InN Core Shell Structured Nanoparticles

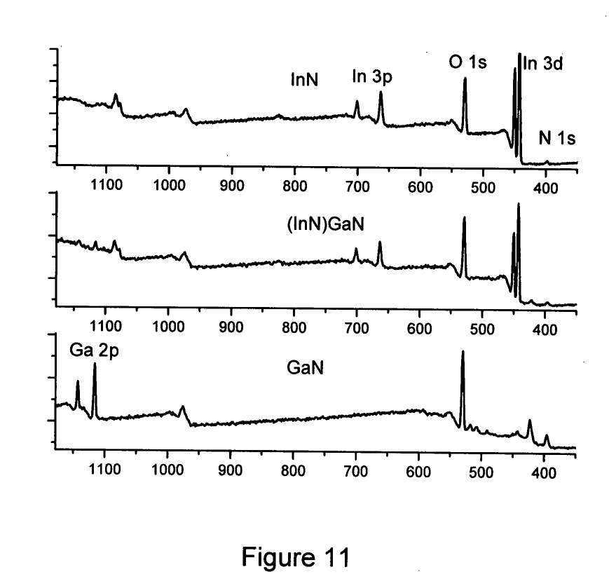

An InN core was synthesized by dissolving 5 g TOPO in 10 mL chloroform in a round bottom flask. After stirring for 5 minutes, 1.11 g (5 mmol) indium chloride and 0.81 g (15 mmol) potassium borohydride were dispersed in the solution, after which 5 mL (36 mmol) triethylamine was added while stirring. After stirring for 30 minutes, the flask was sealed and the solution heated at 200-450° C. for 12 hours. The solution was allowed to cool to room temperature, the cover of the flask was loosened, and the flask was placed in a vacuum chamber to evaporate the solvent. The GaN shell was prepared by dissolving 0.88 g (5 mmol) gallium chloride in 10 mL chloroform, following which the solution was slowly added to the concentrated InN solution while stirring. The flask was sealed and heated at 200-350° C. for 2 hours, following which it was allowed to cool to room temperature. 2 mL of triethylamine was added to the solution, following which the flask was resealed and heated for 12 hours. The solution was allowed to cool to room temperature, following which the dark brown colored (InN)GaN suspension was purified. Comparative XPS spectra of GaN, InN, and (InN)GaN nanoparticles are shown in FIG. 11.

(GaN)InN Core Shell Structures were Synthesized Using Similar Reactions.

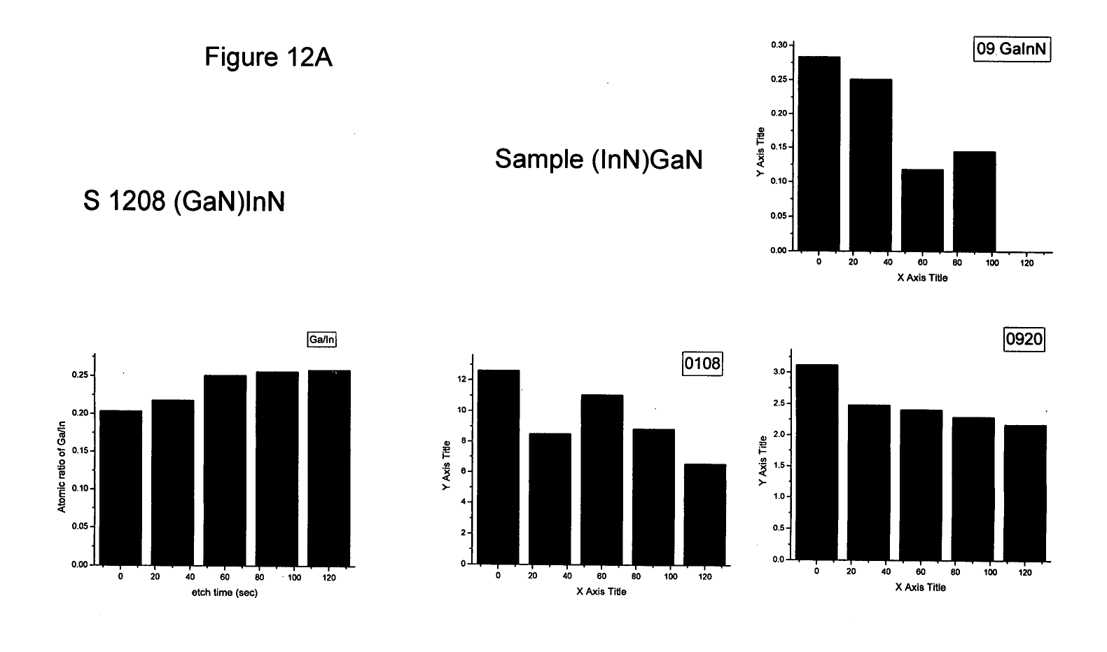

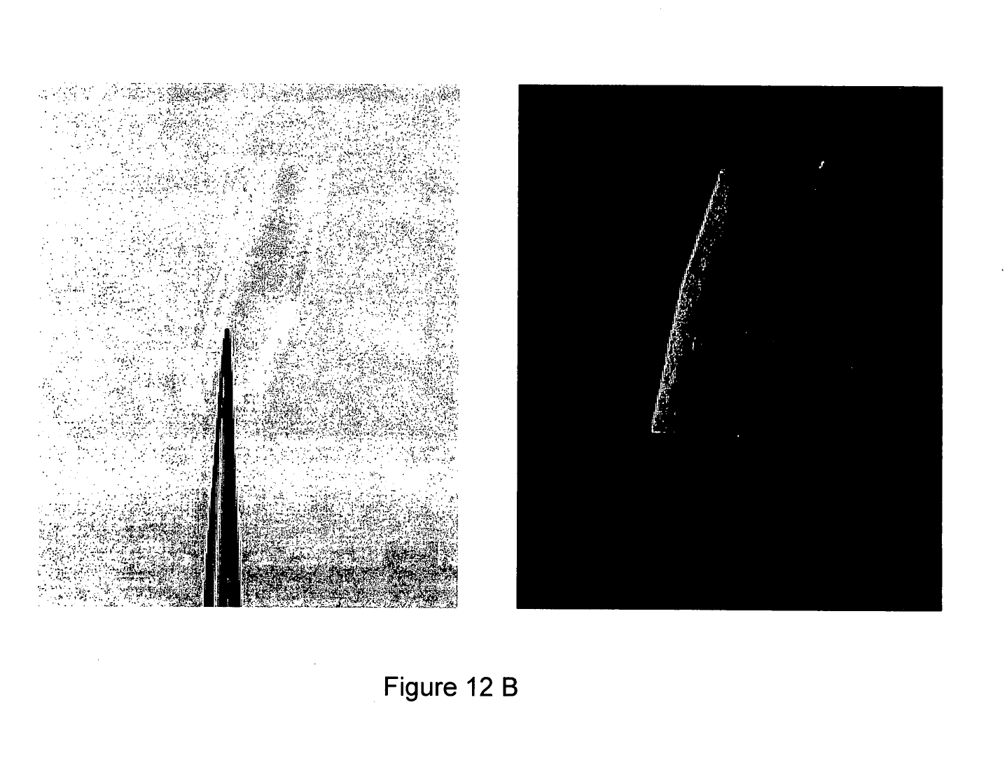

The ion gun from a standard XPS was used to etch both (InN)GaN and (GaN)InN core shell structures. The atomic ratio of Ga to In for various samples is shown in FIG. 12A.

### GaN Nanoparticle Capping

GaN nanoparticles were capped with amino acids. 1 nano-mole of GaN nanoparticles were dispersed into 2 mL of water, then 0.1 mL of an aqueous amino acid solution (0.1 mM/mL) was added. The solution was sonicated for 5 minutes at room temperature and subsequently heated at 80° C. for 12 hours.

GaN nanoparticles were coated with cationic polymers, as described elsewhere for other inorganic nanoparticles (Jaffar, 2004). Briefly, the nanoparticles were diluted to a concentration of 10 nmol/mL and rapidly mixed with an equivolume solution of 10 mg/mL of polyallylamine (PAA, 16 kDa, Sigma). After 20 minutes, excess polymer was removed by spinning the mixture through Amicon separating columns (100 kDa, Millipore) and re-suspending the coated nanoparticles in 0.1 M Tris buffer. Subsequently, the nanoparticle-PAA solution was mixed with 10 mg/mL bovine serum albumin (BSA, Sigma) solution for 20 min and purified twice by spinning through the Amicon separating columns.

### Measurement of Zeta Potential

The electrophoretic mobility and the zeta potential of the nanoparticles were determined using a ZetaPals Analyzer. 100 μL of nanoparticle solution was dispersed in 1.7 mL of pH adjusted water. The pH of the solution was varied by adding concentrated HCl or NaOH to deionized water.

### Production of Water Soluble Nanoparticles

60% DPPC-mPEG2000-COOH was mixed with 1 mL chloroform at room temperature. 1 mL of 1 μM TOPO-capped GaN nanoparticles in chloroform were added and the solvent evaporated overnight. 1 mL water was added and the solution heated to 80° C. for about an hour. The solution was dialyzed for 17 hours against 18 Mohm water using a Spectrapor 3 Membrane (MWCO 3500). The TOPO-capped nanoparticles had a zeta potential of 77 mV, which was reduced to −46 mV after reaction with the DPPC/PEG.

### Nanoparticle Patterning

Patterned substrates were produced using soft microcontact printing of hyaluronic acid (HA) on glass, as reported previously (Jaffar, 2004). Briefly, silicon masters were used to cast polydimethylsiloxane (PDMS) stamps. Glass slides were plasma cleaned for 5 min, spin-coated with 5 mg/mL of HA, brought into conformal contact with the PDMS stamps, and allowed to dry overnight. The stamps were then peeled, and the freshly exposed glass surfaces were rinsed three times in deionized water, producing HA-patterned glass substrates. These patterned substrates were covered for 30 min by a thin film of PAA-coated nanoparticles, and rinsed three times in deionized water. The samples were allowed to dry and imaged using an Olympus microscope (IX51) with a DAPI (ex 360/40, em 460/50) filter.

### Library Screening

Materials screened were MOCVD-grown (0001) GaN thin films grown on c-plane sapphire. Prior to the screening experiments, the surface of the GaN was cleaned in dilute HCl acid for 5 minutes, followed by rinsing in DI H₂O.

All phage display libraries used in screening experiments were obtained from New England BioLabs (NEB, Beverly, Mass.) and were used as received. The specific peptide sequence library emerged in the 3rd round of biopanning for 12 mer M13 phages with GaN target. 10 μL of the original phage library as supplied by NEB were added to a GaN epitaxial surface sealed by an O-ring of 0.5 mL of 50 mM Tris-buffered Saline (TBS) with 0.5% Tween-20 (0.5% TBST) and the selection process proceeded according to the standard method. After phage libraries interacted with the substrate for one hour, the substrate was washed in TBST, followed by elution and quantization of phage bound to the surface via titering. Phage clones isolated were titered to determine phage concentration. Equivalent phage inputs of individual clones were added to individual substrates in 1 mL of 0.5% TBST. Several clones were tested in parallel. Clones were interacted with a substrate for an hour. The substrates were then washed three times. Bound phage were then isolated using the standard acidic elution method as used when screening. The eluate was then titered to determine the concentration and hence binding activity of one clone relative to another. Phage DNA was precipitated according to the standard protocols supplied by NEB and DNA sequencing was performed by DNA sequencing facilities to decipher the possible material-specific binding motifs of the high affinity clones.

### Viral Film Preparation.

0.5 mL GaN nanoparticles solutions ranging in concentration form 0.5 to 3 μM for pIII fusions and 100 μM for pVIII fusions was added to 1 mL of a solution of M13 phage clones having a concentration of 10-150 mg/mL and shaken for eight hours. The suspensions were allowed to dry in a desiccator for 2 weeks. A film produced using 80 mg/mL pIII-phage and 1.5 μM GaN particles is shown as photographed under room light and UV light in FIG. 12B.

### Polarized Optical Microscopy (POM).

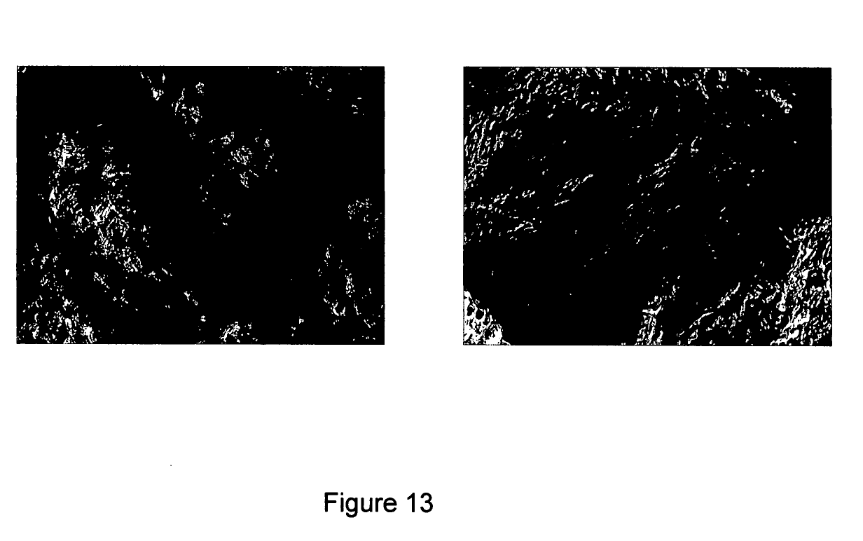

POM images of the nanoparticle/phage hybrid films were obtained using an Olympus polarized optical microscope (IX51). Micrographs were taken using a charge coupled device (CCD) digital camera. The optical activity was also observed by changing the angles between the polarizer and analyzer (FIG. 13).

### Atomic Force Microscopy (AFM).

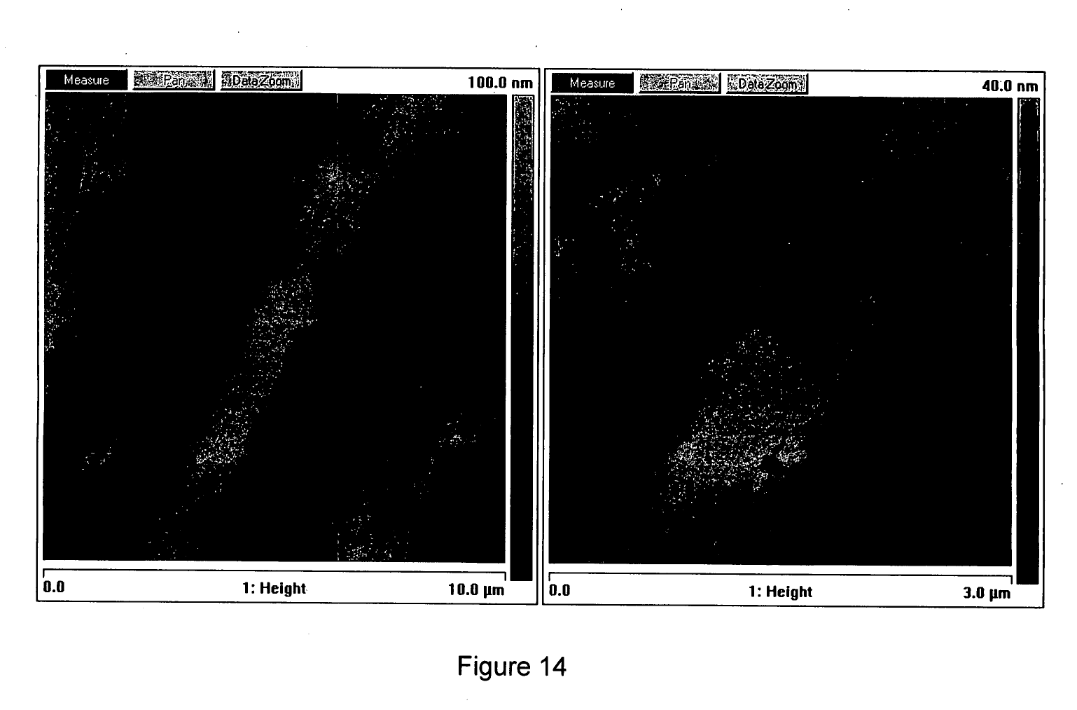

An atomic force microscope (Digital Instruments) was used to study the surface morphologies of the viral film. The images were taken in air using tapping mode. The AFM probes were etched silicon with 125 pm cantilevers and spring constants of 20-100 Nm driven near their resonant frequency of 250-350 kHz (FIG. 14).

### Nanoparticle-Virus Hybrid Nanowires

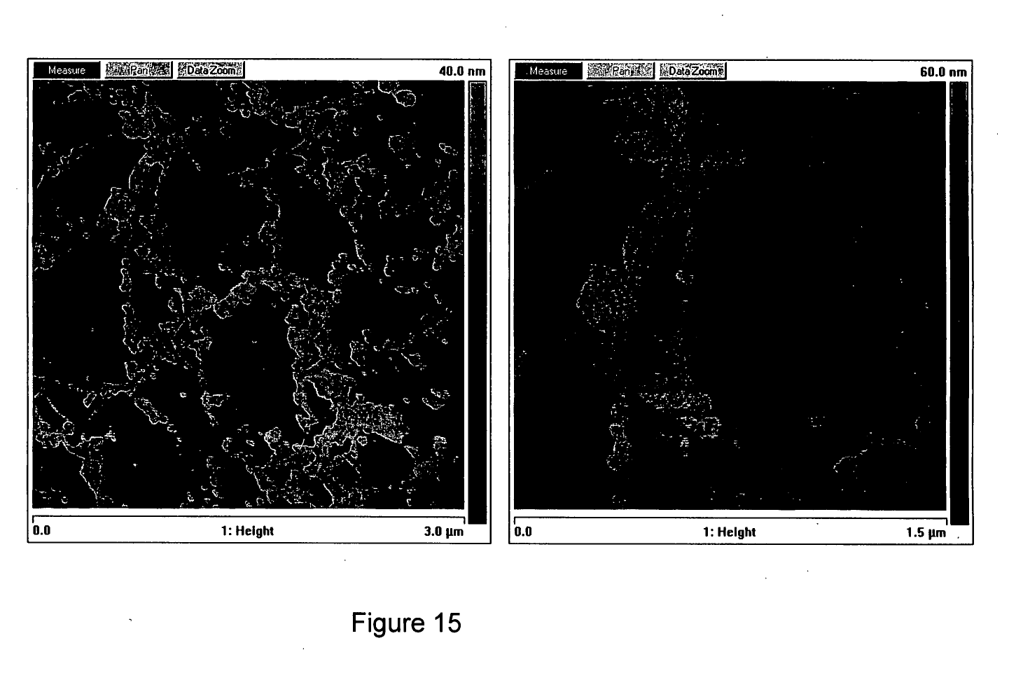

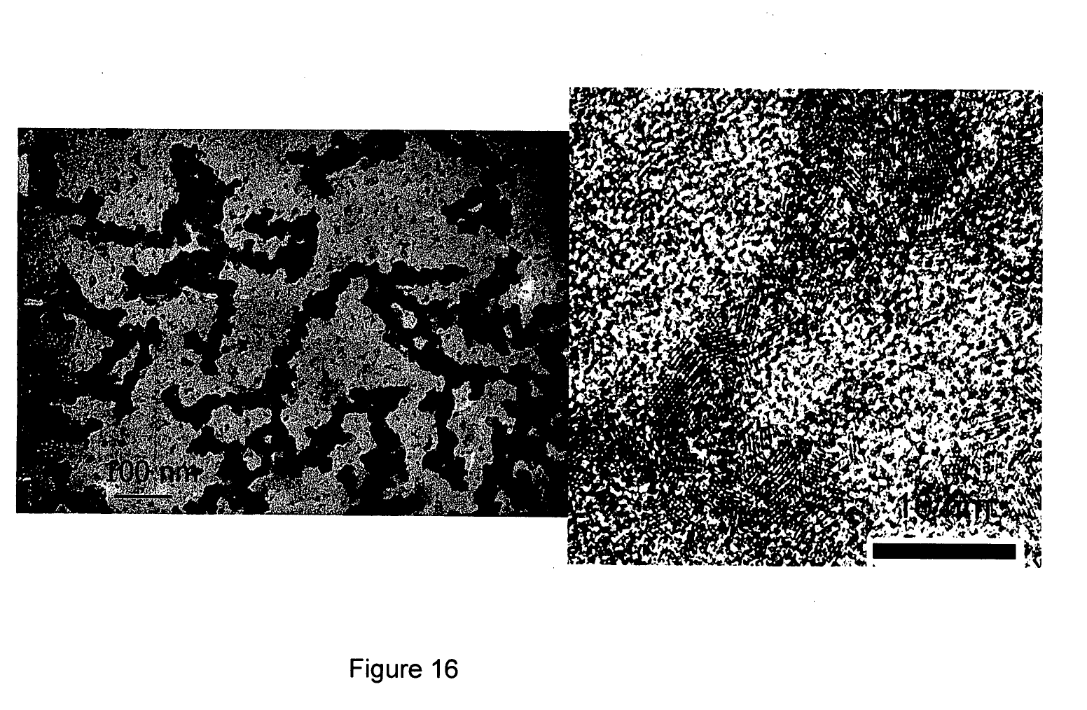

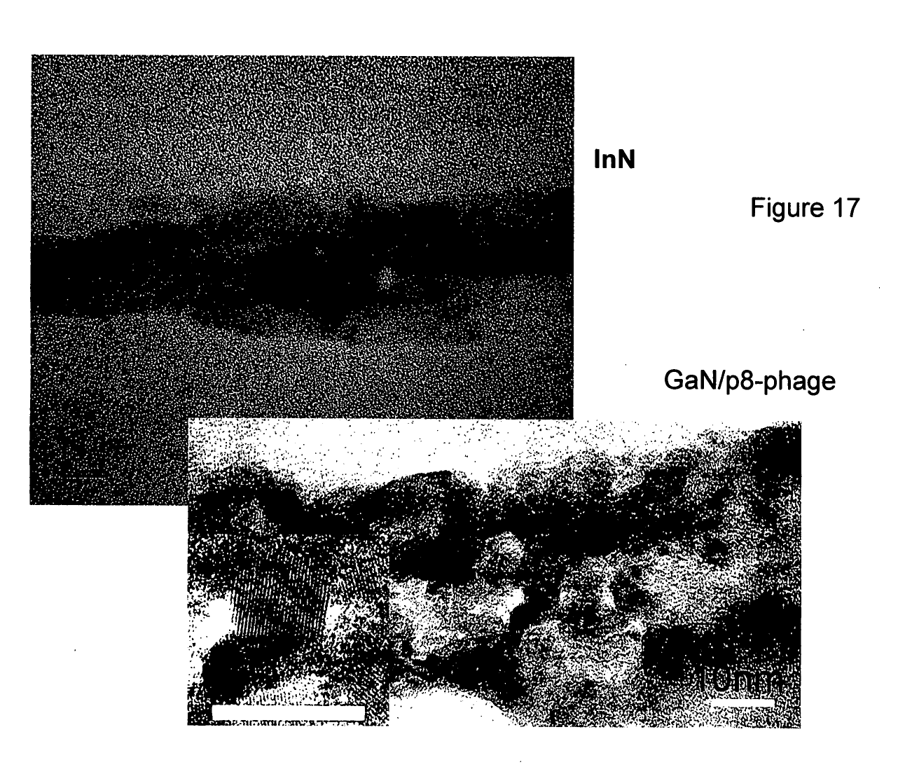

0.5 mL of 10¹⁰ pfu pVIII hybrid phage (Val-Ser-Pro-Ser-Gly-Thr-Pro-Glu) was combined with a large excess (e.g., over 2700 times, about 25 nM) of GaN, GaInN, or InN nanoparticles and vortexed overnight at room temperature. The resulting nanowires were imaged by TEM and AFM and are shown in FIGS. 15, 16, and 17.

### Cellular Uptake

We studied the interaction of GaN nanoparticles with COS-7 monkey kidney epithelium cells. COS-7 cells cultured in DMEM supplemented with 10% FBS and 1% PSAB were grown overnight. They were incubated for 2 hours in serum-free medium supplemented with 50 pmol/mL of uncoated GaN nanoparticles. After treatment, the nanoparticle-medium was replaced with complete medium, and cells were allowed to grow for 20 hours. Subsequently, they were incubated in 50 nM Lysotracker (Red DND-99, Molecular Probes) for 20 min, and imaged using an Olympus microscope (IX51) with DAPI (ex/em) and TRITC (ex 535/50, em 610/75 nm) filters.

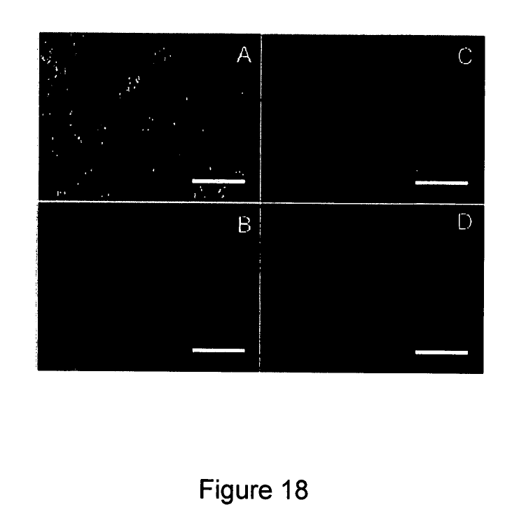

FIG. 18A is the phase contrast image of the cells. FIG. 18B is an image through the DAPI filter that shows the GaN nanoparticles associated with the cells.

FIG. 18C is imaged with a TRITC filter and highlights the acidic organelles (endosomes and lysosomes), as revealed by lysotracker staining. FIG. 18D is a merged image of the GaN nanoparticles and endosomes, to determine co-localization.

The morphologies of the GaN treated cells (FIG. 18A), as determined by the phase contrast images, indicate that they are healthy. The cells are elongated and sprout processes, while their cellular and nuclear membranes are intact. They do not curl up, nor are they fragmented. Since the cells appear to be metabolically active despite being exposed to GaN nanoparticles, these particles are likely to be non-cytotoxic. Furthermore, since the GaN bond is very stable, it is unlikely to be photo-oxidized and cause subsequent cell death, as has been observed for CdS nanoparticles (Derfus, 2004). Treated cells were maintained in culture for up to 72 hours without observation of significant nanoparticle-induced cytotoxicity. Photo-illumination of the treated cells results in bright blue fluorescence (FIG. 18B) characteristic of the GaN nanoparticles. All the cells visualized had some degree of detectable fluorescence. In most of the cells, fluorescence was evenly distributed throughout the cytoplasmic area but conspicuously absent from the nuclear region. This implies that, if the nanoparticles were being internalized into the cell, they were unable to penetrate into the nucleus. To determine whether the nanoparticles were adsorbed onto the surface or internalized within the cell, the nanoparticles were co-localized with lysotracker, an acidic organelle dye (FIG. 18C), and visualized. There is complete overlap between the images (FIG. 18D), indicating that nanoparticles have been incorporated into the cell through endocytosis. These internalized nanoparticles remain vesicle-bound and are not released into the cytoplasm of cells.

Other embodiments of the invention will be apparent to those skilled in the art from a consideration of the specification or practice of the invention disclosed herein. It is intended that the specification and examples be considered as exemplary only, with the true scope and spirit of the invention being indicated by the following claims.

What is claimed is:

1. A method of producing colloidal III-V semiconductor crystals, comprising:
reacting a solution comprising at least one source material including a Group III element, a source material including a Group V element, and a reducing agent for a predetermined time period at a predetermined temperature,
wherein the source material is a salt of a Group III element, a mono, di, or trialkyl compound of a group V element, or a chelate of a Group III element with a mono-, di-, or trialkyl compound of a group V element.

2. The method of claim 1, wherein the at least one Group III element is gallium, indium, or aluminum.

3. The method of claim 1, wherein the at least one Group III element is gallium, indium, aluminum, or boron.

4. The method of claim 1, wherein the Group V element is nitrogen.

5. The method of claim 1, wherein the Group V element is nitrogen, phosphorus, arsenic, or antimony.

6. The method of claim 1, wherein the predetermined temperature is about 100° C. to about 450° C.

7. The method of claim 1, wherein the predetermined temperature is about 180° C. to about 450° C.

8. The method of claim 1, wherein the predetermined time period is between 8 and 16 hours.

9. The method of claim 1, wherein the solution further comprises a source material of a rare earth element or a transition metal, wherein the a salt of the rare earth or transition metal element or a chelate of the rare earth or transition metal element with a mono-, di-, or trialkyl compound of the group V element.

10. The method of claim 1, wherein the solution includes source materials for a first Group III element and a second Group III element.

11. The method of claim 10, wherein the ratio of the first Group III element to the second Group III element is between 1:99 and 99:1.

12. The method of claim 11, wherein the ratio of the first Group III element to the second Group III element is about 95:5.

13. The method of claim 11, wherein the ratio of the first Group III element to the second Group III element is about 90:10.

14. The method of claim 11, wherein the ratio of the first Group III element to the second Group III element is about 80:20.

15. The method of claim 11, wherein the ratio of the first Group III element to the second Group III element is about 70:30.

16. The method of claim 11, wherein the ratio of the first Group III element to the second Group III element is about 60:40.

17. The method of claim 11, wherein the ratio of the first Group III element to the second Group III element is about 50:50.

18. The method of claim 1, wherein the source material for the Group III element and the source material for the Group V element are the same material.

19. The method of claim 1, wherein the solution further comprises a solvent, wherein the source material for the Group V element is the solvent.

20. The method of claim 1, wherein the solution further comprises a solvent, wherein the solvent is triethylamine.

21. The method of claim 1, wherein the solution further comprises a solvent, wherein the solvent is acetonitrile, chloroform, benzene, paraffin oil, and naphthalene

22. The method of claim 1, wherein the solution further includes a capping agent.

23. The method of claim 22, wherein the capping agent is TOPO, polyallylamine, hyaluronic acid, acetamidine hydrochloride, cetyltrimethyl ammonium bromide, benzalkonium chloride, poly(vinylsulfonic acid), linear and branched poly(ethylene imine) PEI, polyallylamine HCl (PAH), polylysine, chitosan, poly(diallydimethylammonium chloride) (PDAC), a polysaccharide, a polymer of positively charged amino acids, polyaminoserinate, hyaluronan, polymalic acid, a polyimide, phenylalanine, histidine, hexahistidine, serine, proline, a polymer of negatively charged or acidic amino acids, a phospholipid, a PEG-derivitized phospholipid, a polynucleotide, or an amino acid oligomer.

24. The method of claim 22, wherein the capping agent is A-R-X, wherein A is thiol, phosphine, phosphine oxide, amine, amide oxide, sulfonate, carbonate, or carboxylate, R is straight or branched alkane optionally comprising amide, ketone, ether, or aryl, and X is hydroxyl, amine, amide, carboxylate, sulfonate, phosphate, or ammonium.

25. The method of claim 22, wherein the Group III element is gallium, the Group V element is nitrogen, and the capping agent is Ser-Ser-Phe-Ser-Asn-Val-Thr-Ser-Gly-Thr-Gln-Lys (SEQ ID NO: 1), Lys-Leu-His-His-Ser-Pro-Pro-Pro-Pro-Phe-Val-Phe (SEQ ID NO: 2), or Val-Ser-Pro-Ser-Gly-Thr-Pro-Glu (SEQ ID NO: 3).

26. The method of claim 1, further comprising:
condensing the solution by removing at least a portion of the solvent; and
heating the remaining product at a temperature between about 300° C. and 450° C. for about 8 to about 16 hours.

27. The method of claim 1, further comprising recovering III-V nanoparticles from the solution, suspending the nanoparticles in a solvent with a source material including a predetermined Group III element and a source material including a predetermined Group V element, and holding the suspension at a predetermined temperature for a predetermined period of time, wherein a layer of a semiconductor material including the Group III element and the Group V element forms on the nanoparticle.

28. The method of claim 1, further comprising recovering the nanoparticles and covalently or non-covalently attaching a biologically active agent to the nanoparticles.

29. The method of claim 1, further comprising recovering the nanoparticles and covalently or non-covalently conjugating them to a targeting agent.

30. A method of patterning nanoparticles on a surface, comprising,
producing colloidal III-V semiconductor crystals according to the method of claim 1;
capping the III-V semiconductor crystals with a material having a predetermined charge;
providing a substrate having a charged material patterned thereon, the charged material having a charge opposite that of the predetermined charge; and
incubating the substrate with the capped III-V semiconductor crystals.

31. The method of claim 30, wherein providing a substrate comprises patterning the charged material on the substrate.

32. The method of claim 30, wherein the charged material is a SAM-forming material or one of TOPO, polyallylalanine, hyaluronic acid, acetamidine hydrochloride, cetyltrimethyl ammonium bromide, benzalkonium chloride, poly(vinylsulfonic acid), linear and branched poly(ethylene imine) PEI, polyallylamine HCl (PAH), polylysine, chitosan, poly(diallydimethylammonium chloride) (PDAC), polysaccharides, polymers of positively charged amino acids, polyaminoserinate, hyaluronan, polymalic acid, polyimides, polymers of negatively charged or acidic amino acids, and polynucleotides.

33. A core shell structure comprising a core of a first III-V semiconductor material including a first Group III element and a first Group V element and a layer of a second III-V semiconductor material including a second Group III element and a second Group V element.

34. The core shell structure of claim 33, wherein the first and second Group III elements are the same or the first and second Group V elements are the same.

35. The core shell structure of claim 33, wherein the first and second Group III elements are not the same and wherein the first and second Group V elements are not the same.

36. The core shell structure of claim 33, further including a capping layer disposed on the surface of the core shell structure.

37. The core shell structure of claim 33, wherein the core is substantially GaN and the shell is substantially InN.

38. The core shell structure of claim 33, wherein the core is substantially InN and the shell is substantially GaN.

39. The core shell structure of claim 33, wherein each of the core and shell is independently selected from AlP, AlAs, AlSb, AIN, GaN, GaP, GaAs, GaSb, InP, InAs, InSb, and InN.

40. The core shell structure of claim 33, wherein the thickness of the shell is about the same as the radius of the core.

41. The core shell structure of claim 33, wherein the ratio of the shell thickness and the core radius is between about 1:1 and 1:5.

42. The core shell structure of claim 33, wherein the ratio of the shell thickness and the core radius is between about 1:1 and 1:4.

43. The core shell structure of claim 33, wherein the ratio of the shell thickness and the core radius is between about 1:1 and 1:3.

44. The core shell structure of claim 33, wherein the ratio of the shell thickness and the core radius is between about 1:1 and 1:2.

45. A III-V semiconductor nanoparticle comprising a first Group III element and a second Group III element in a predetermined ratio and a Group V element.

46. The III-V semiconductor nanoparticle of claim 45, wherein the predetermined ratio is between about 1:99 and about 70:30.

47. The III-V semiconductor nanoparticle of claim 45, wherein the predetermined ratio is about 95:5.

48. The III-V semiconductor nanoparticle of claim 45, wherein the predetermined ratio is about 90:10.

49. The III-V semiconductor nanoparticle of claim 45, wherein the predetermined ratio is about 80:20.

50. The III-V semiconductor nanoparticle of claim 45, wherein the nanoparticle diameter is between about 2 and about 15 nm.

51. The III-V semiconductor nanoparticle of claim 45, wherein the nanoparticle diameter is between about 15 and about 30 nm.

52. The III-V semiconductor nanoparticle of claim 45, wherein the nanoparticle diameter is between about 5 and about 7 nm.

53. A population comprising a plurality of the III-V semiconductor nanoparticles of claim 45, wherein the variation of the particle diameter is about 15% or less.

54. A population comprising a plurality of the III-V semiconductor nanoparticles of claim 45, wherein the variation of the particle diameter is about 10% or less.

55. A population comprising a plurality of the III-V semiconductor nanoparticles of claim 45, wherein the variation of the particle diameter is about 5% or less.

56. A population comprising a plurality of the III-V semiconductor nanoparticles of claim 45, wherein the population is a colloidal solution of III-V semiconductor nanoparticles.

57. A colloidal solution of Group III-nitride semiconductor crystals.

58. The colloidal solution of claim 57, wherein the Group III element is one or two of Ga, Al, and In.

59. The colloidal solution of claim 57, wherein the Group III element is one or two of Ga, Al, In, and B.

60. The colloidal solution of claim 57, wherein the semiconductor crystals are between about 2 and about 15 nm in diameter.

61. The colloidal solution of claim 57, wherein the semiconductor crystals are between about 15 and about 30 nm in diameter.

62. The colloidal solution of claim 57, wherein the semiconductor crystals include a capping agent.

63. The colloidal solution of claim 57, wherein the semiconductor crystals are conjugated to a biologically active agent.

64. The colloidal solution of claim 57, wherein the semiconductor crystals are water soluble.

65. A colloidal solution of substantially spherical III-V semiconductor crystals, wherein the variation in particle diameter of the crystals is about 15% or less.

66. The colloidal solution of claim 65, wherein the variation in particle diameter is about 10% or less.

67. The colloidal solution of claim 65, wherein the variation in particle diameter is about 5% or less.


SEQUENCE LISTING (5 sequences)

SEQ ID NO: 1 - protein, 12 aa, Artificial Sequence
  12 amino acid peptide identified to bind to GaN
SSFSNVTSGTQK

SEQ ID NO: 2 - protein, 12 aa, Artificial Sequence
  12 amino acid peptide identified to bind to GaN
KLHHSPPPPFVF

SEQ ID NO: 3 - protein, 8 aa, Artificial Sequence
  8 amino acid peptide identified to bind to GaN
VSPSGTPE

SEQ ID NO: 4 - protein, 12 aa, Artificial Sequence
  12 amino acid peptide identified to bind to GaN
YPTHHAHTTPVR

SEQ ID NO: 5 - protein, 12 aa, Artificial Sequence
  12 amino acid peptide identified to bind to GaN
TSDIKSRSPHHR